(19) United States
(12) Patent Application Publication
(10) Pub. No.: US 2024/0181092 A1
(43) Pub. Date: Jun. 6, 2024

(54) DIAGNOSTIC METHODS OF PROSTATE CANCER

(71) Applicant: NOVARTIS AG, Basel (CH)

(72) Inventors: Ana CATAFAU, Geneva (CH); Elisabet PARERA PIELLA, Marseille (FR); Beilei HE, Geneva (CH); Euloge KPAMEGAN, Emeryville, CA (US)

(21) Appl. No.: 18/552,760

(22) PCT Filed: Apr. 1, 2022
(86) PCT No.: PCT/EP2022/058754

§ 371 Date: Sep. 27, 2023

(30) Foreign Application Priority Data
Apr. 2, 2021 (EP) 21305432.3

(51) Int. Cl. A61K 51/04; A61K 51/12

(52) U.S. Cl. CPC A61K 51/0402; A61K 51/0497; A61K 51/121

(57) ABSTRACT

The present disclosure relates to the field of diagnostic methods, and more particularly prostate cancer imaging. In particular, the disclosure relates to a radiopharmaceutical PSMA-binding compound for use in determining the presence and/or localization of PSMA-positive tumors in a subject in need thereof, wherein said subject has been diagnosed with biochemical recurrence, particularly after radical prostatectomy or radiotherapy, and wherein said radiopharmaceutical compound is the compound of formula (III).

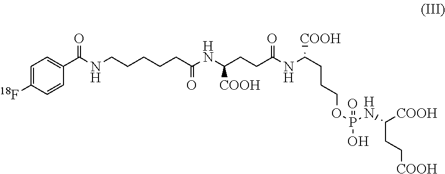 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

### TECHNICAL FIELD

The present disclosure relates to the field of diagnostic methods, and more particularly prostate cancer imaging.

### BACKGROUND ART

Prostate-specific membrane antigen is a transmembrane protein, also known as folate hydrolase or glutamate carboxypeptidase II. From all the known PSMA-overexpressing tumors, prostate cancer is the one in which the role of PSMA has been most extensively studied. Prostate cancer remains the cancer with the second highest mortality in the United States (US), and the third leading cause of cancer-related death in Europe in men (Siegel RL, Miller KD, Jemal A (2017) Cancer Statistics, 2017. CA Cancer J Clin; 67(1):7-30, Malvezzi M, Carioli G, Bertuccio P, et al (2019) European cancer mortality predictions for the year 2019 with focus on breast cancer. Ann Oncol; 30(5):781-7). It also remains the most diagnosed cancer with an estimated increase of 9,960 new cases with a total of 174,650 in 2019 (Siegel RL, Miller KD, Jemal A (2018) Cancer Statistics, 2018. CA Cancer J Clin; 68(1):7-30, Siegel RL, Miller KD, Jemal A (2019) Cancer statistics, 2019. CA Cancer J Clin; 69(1):7-34). Most of the diagnosed cases are in more developed regions due to the use of PSA testing, but there is only modest variation in mortality rates globally which is driven by metastatic, and often castration-resistant disease (Bray F, Ren JS, Masuyer E, et al (2013). Int J Cancer; 132:1133-45). Subsequent treatment is multifaceted and may involve observation, surgery (prostatectomy), radiation therapy (external beam or brachytherapy), hormonal therapy, chemotherapy. The differential expression of PSMA from tumor to non-tumor tissue has resulted in numerous targeted strategies involving both disease localization using PSMA-PET imaging as well as therapeutic intervention. Correct identification of disease location and extent determines treatment decisions for patients with prostate cancer. Identification of distant metastatic disease at the early stages of prostate cancer is thus important in planning prostate cancer management.

Up to 40% of the patients with prostate cancer develop biochemical recurrence (BCR) within 10 years after initial treatment (Isbarn et al 2010. BJU Int; 106:37-43). Usually an increase of the PSA-level precedes a clinically detectable recurrence by months to years (Van Poppel et al 2006, (EORTC 30001). Eur J Cancer; 42:1062-7). However, it cannot differentiate between local, regional or systemic disease with the necessary precision that is essential for further disease management (Bott 2004. Prostate Cancer and Prostatic Dis; 7:211-6).

It is relevant, therefore, to detect smaller and distant lesions as early as possible, in particular in patients with biochemical recurrence.

The usual diagnostic tools for prostate cancer include PSA testing, digital rectal palpation, transrectal ultrasound, prostate biopsy, and histopathologic examination (Schwarzenböck S, Souvatzoglou M, Krause BJ (2012). Theranostics; 2(3):318-30; Smith RA, Andrews K, Brooks D, et al (2016) Cancer screening in the United States, 2016: A review of current American Cancer Society guidelines and current issues in cancer screening. CA Cancer J Clin; 66(2):95-114; Prasad V, Steffen IG, Diederichs G, et al (2016). Mol Imaging Biol; 18:428-36). Additionally, further imaging techniques such as Magnetic Resonance Imaging (MRI), bone scintigraphy, CT, and [18F]Fluorodeoxyglucose (FDG), [18F]Choline, [11C]Choline and the more recently approved [18F]Fluciclovine (Nanni C, Zanoni L, Pultrone C, et al (2016) (¹⁸)F-FACBC (anti1-amino-3-(18)F-fluorocyclobutane-1-carboxylic acid) versus (11)C-choline PET/CT in prostate cancer relapse: results of a prospective trial. Eur J Nucl Med Mol Imaging; 43:1601-10, Odewole OA, Tade FI, Nieh PT, et al (2016) Recurrent prostate cancer detection with anti-3-[(¹⁸)F]FACBC PET/CT: comparison with CT. Eur J Nucl Med Mol Imaging; 43:1773-83) PET/CT are used for staging primary prostate cancers and restaging biochemical recurrences (Schwarzenböck S, Souvatzoglou M, Krause BJ (2012) Choline PET and PET/CT in Primary Diagnosis and Staging of Prostate Cancer. Theranostics; 2(3):318-30).

CT and MRI are the standard of care imaging procedures for measuring tumors at baseline and lesions selected for response assessment as per Response Evaluation Criteria in Solid Tumors (RECIST) 1.1 (Eisenhauer EA, Therasse P, Bogaerts J, et al (2009) New response evaluation criteria in solid tumours: revised RECIST guideline (version 1.1). Eur J Cancer; 45:228-47). However, these imaging modalities have shown limited yielding in the staging of pelvic lymph nodes in patients with prostate cancer.

Therefore, more sensitive and accurate imaging tests than the currently available standard-of-care examinations are needed. Novel PET radiotracers promise to overcome this limitation. PET imaging is an enticing choice as it offers the potential to both stage patients and provide insight into tumor biology. Among the various PET probes available, ⁶⁸Ga-labeled ligands of the PSMA were associated with unprecedented accuracy and effect on treatment in several meta-analyses of retrospective studies (Perera M, Papa N, Christidis D, et al (2016). Eur Urol; 70:926-37; Han S, Woo S, Kim YJ, et al (2018). Eur Urol; 74:179-90, Von Eyben FE, Picchio M, von Eyben R, et al (2018). Eur Urol Focus: 4:686-93). In particular, several head to head comparison studies have been performed on the uptake of [¹⁸F]Fluciclovine vs [⁶⁸Ga]Ga-PSMA-11 for localization of prostate cancer tumors in patients with biochemical recurrence. Detection rates were superior for [⁶⁸Ga]Ga-PSMA-11 vs [¹⁸F]Fluciclovine after radical prostatectomy in patients with PSA <2.0 ng/ml on per-patient and per-region basis (Calais et al 2018 Potential Impact of 68Ga-PSMA-11 PET/CT on the Planning of Definitive Radiation Therapy for Prostate Cancer. J Nucl Med; 59(11):1714-21). However, a larger prospective head-to-head comparison study on [¹⁸F]Fluciclovine vs [⁶⁸Ga]Ga-PSMA-11 in patients with biochemical recurrence of prostate cancer found no statistical differences in the overall detection rate for prostate cancer recurrence between the two different radioligands (Pernthaler et al 2019 A Prospective Head-to-Head Comparison of ¹⁸F-Fluciclovine With 68Ga-PSMA-11 in Biochemical Recurrence of Prostate Cancer in PET/CT. Clin Nucl Med; 44(10):e566-e73).

Hence, there is still a need in identifying radioligands capable of detecting and localising tumors in prostate cancer patients with biochemical recurrence with improved detection rates and/or localisation.

[¹⁸F]CTT1057 is a promising novel PSMA-targeting ¹⁸F-labeled PET imaging agent (WO2014143736). Unlike most other PSMA agents labelled with either ⁶⁸Ga or ¹⁸F (e.g. [⁶⁸Ga]Ga-PSMA-11, [¹⁸F]PSMA1007, [¹⁸F]DCFPyL) which share a urea backbone, [¹⁸F]CTT1057 is based on a phosphoramidate scaffold that irreversibly binds to PSMA with high nanomolar affinity, which may account for a higher and prolonged tumor uptake (Behr SC, Aggarwal R, VanBrocklin HF, et al (2019) Phase I Study of CTT1057, an ¹⁸F-Labeled Imaging Agent with Phosphoramidate Core Targeting Prostate-Specific Membrane Antigen in Prostate Cancer. J Nucl Med; 60(7):910-6).

The [¹⁸F]CTT1057 Phase-I study in 20 prostate cancer patients (n=5 primary staging and n=15 metastatic-castration resistant prostate cancer (mCRPC)) has shown an acceptable safety profile without any radiotracer-related adverse reactions. The Phase-I study also demonstrated that metastatic lesions are detected with higher sensitivity on [¹⁸F]CTT1057 imaging than on conventional imaging (Behr et al 2019). Another smaller study showed that the image quality on [¹⁸F]CTT1057 PET imaging was qualitatively similar to that obtained on [⁶⁸Ga]Ga-PSMA-11 PET (Behr S, Aggarwal R, Flavell R, et al (2017) [abstract]. J Nucl Med; 58 Suppl 1:733A).

### SUMMARY

The present disclosure provides novel methods of use of PET imaging agents for detection and localization of PSMA positivity in patients diagnosed of biochemical recurrence, in particular in patients with prostate cancer.

In particular, an objective of the present disclosure is to provide methods of detecting PSMA-positive tumors using PET imaging agents with a high affinity towards a target on PSMA-expressing cancer cells, preferably prostate cancer cells providing very high tumor-to-background ratios.

It is another objective of the present disclosure to provide methods for identifying very small volume sites of disease.

It is another objective of the present disclosure to provide methods of detecting PSMA-positive tumors, preferably prostate cancer tumors, with PET imaging agents having a biodistribution favorable to the detection of typical sites of disease such as the prostate bed, pelvic lymph nodes, and bone.

It is another objective of the present disclosure to provide methods of detecting PSMA-positive tumors, preferably prostate cancer tumors, which work reliably among a large variety of different clinical contexts including initial staging, restaging at the time of biochemical recurrence, radiation or surgery planning.

It is another objective of the present disclosure to provide methods of detecting PSMA-positive tumors, preferably prostate cancer tumors, with imaging agents having a high radiochemical yield enabling for a high throughput of patients.

The present disclosure thus relates to a radioligand imaging agent, for use in determining the presence and/or localization of PSMA-positive tumors in a subject, wherein said subject has been diagnosed with biochemical recurrence, and wherein said radioligand imaging agent is a PSMA-binding compound comprising a phosphoramidate group and a [¹⁸F]-fluoro group.

The disclosure also relates to a solution for injection or infusion, which is an aqueous solution comprising a PSMA-binding compound comprising a phosphoramidate group and a [¹⁸F]-fluoro group, in a concentration providing a volumetric radioactivity between 150 and 1000 MBq/mL, for example about 370 MBq/mL, and one or more pharmaceutically acceptable excipients.

Also disclosed herein are methods for determining the presence and/or the localization of PSMA positive tumors in a subject, preferably a subject with prostate cancer, wherein said subject has been diagnosed with biochemical recurrence, said method comprising
[i] administering to said subject, an effective dose of a radioligand imaging agent as defined below,
[ii] imaging said subject by PET scan, either PET/CT or PET/MRI scan,
[iii] analysing the image obtained by the PET scan, thereby determining the presence and/or the localization of PSMA positive tumors in said subject.

### DETAILED DESCRIPTION

### Definitions

As used herein, the term “PSMA-positive tumor” refers to a tumor lesion which can be detected with a tracer compound comprising a PSMA-binding moiety, typically a radioligand imaging agent such as the ¹⁸F radiolabeled PSMA-binding compound of formula (I), (II) or (III) as described below.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “effective dose” of a radioligand imaging agent for use according to the methods of the disclosure refer to an amount of the imaging agent that is sufficient for determining the presence or localisation of PSMA-positive lesions in a patient from an imaging study using said imaging agent. In particular, in specific embodiments, the presence and/or localisation is more reliably determined with the methods of the present disclosure than conventional imaging methods. An effective dose may be determined by the radioactivity of the injected solution at injection time. The radioactivity of the injected solution may be derived from the measurement of the volumetric radioactivity of the solution for injection at a reference time, typically, after production of the solution for injection, also referred as the “calibration time”. The physician will adjust the volume to be injected based on the estimated volumetric radioactivity at injection time and the known volumetric radioactivity at calibration time.

Accordingly, as used herein, the term “calibration time” when referring to the radioactivity of the volumetric radioactivity of a composition refers to the radioactivity measured at a reference date and time, for example, within 60 minutes after the product manufacturing.

“Radiochemical purity”: is that percentage of the stated radionuclide that is present in the stated chemical or biological form. Radiochromatography methods, such as HPLC method or Thin Layer Chromatography method (TLC), are commonly accepted methods for determining radiochemical purity in the radiopharmacy. In specific embodiments, the radiochemical purity of the radioligand imaging agent is superior or equal to 95%.

As used herein, “aqueous solution” refers to a solution of one or more solute in water. The term “aqueous solution” may also refer to hydroalcoholic solution, including water and an alcohol, preferably ethanol, for example alcohol is comprised between 0% and 20%, preferably between 0% and 10%, for example between 2% and 8%, and more preferably about 5%.

The term “about” or “ca.” has herein the meaning that the following value may vary for ±20%, preferably ±10%, more preferably ±5%, even more preferably ±2%, even more preferably ±1%.

As used herein, the term “amino acids” relates to organic compounds comprising at least one amino group and at least one carboxylic acid group, and includes both natural and non-natural amino-acids.

As used herein, the term “heteroalkylene” refers to a divalent heteroalkyl, which is a straight or branched hydrocarbon chain consisting of 1 to 35 carbon atoms, preferably 1 to 20, and from 1 to 15 heteroatoms selected from the group consisting of O, N and S, and wherein the nitrogen and sulfur atoms may optionally be oxidized (for example: a sulfoxide or a sulfone) and the nitrogen heteroatom may optionally be quaternized. The heteroatom(s) O, N and S may be placed at any interior position of the heteroalkylene group and at either or both of the chain termini.

### The Radioligand Imaging Agent for use in the Methods of the Disclosure

The radioligand imaging agent for use according to the present disclosure is a PSMA-binding compound comprising at least a phosphoramidate group and a [¹⁸F]-fluoro group, or any pharmaceutically acceptable salts thereof.

¹⁸F radiolabeled PSMA-binding compounds have been described in the art, and include those described in WO2013173583 or WO2014143736.

In certain embodiments, the radioligand imaging agent for use according to the present disclosure is a PSMA-binding compound of formula (I):

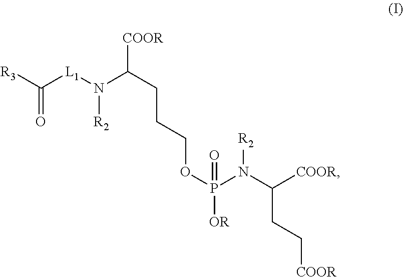

or pharmaceutically acceptable salts thereof wherein
each R is independently hydrogen or a protecting group (e.g t-butyl or benzyl),
each R2 is independently hydrogen or C1-C6 alkyl,
R3 is a phenyl or pyridyl, each substituted with [¹⁸F]-fluoro group and optionally substituted with a second group selected from the group consisting of halo, cyano and nitro,
and L1 is a linker. preferably comprising one or more groups selected from one or more amino-acids, C1-C18 alkylene, and heteroalkylene comprising 1 to 35 carbon atoms and 1 to 15 heteroatoms, said heteroalkylene being optionally substituted by one or more substituents selected from oxo and C1-C6 alkyl, and more preferably L1 is a linker selected from 1 to 6 amino-acids.

In certain embodiments, the radioligand imaging agent for use according to the present disclosure, is a PSMA-binding compound of formula (II):

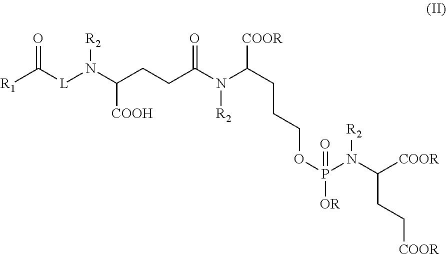

or any pharmaceutically acceptable salts thereof, wherein
L is a linker comprising a moiety of the formula —NH—CH₂CH₂—(OCH₂CH₂—)y—C(O)— or a group of the formula

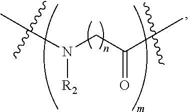

wherein y is 1, 2, 3, 4, 5, 6, 7, 8, 9, 10, 11, or 12:
m is 1, 2, 3, or 4;
Each n is independently 1, 2, 3, 4, 5, 6, 7, 8, 9, 10, 11, or 12;
R1 is a phenyl or pyridyl, each substituted with [¹⁸F]-fluoro group and optionally substituted with a second group selected from the group consisting of chloro and cyano; and
Each R2 is independently hydrogen or C1-C6 alkyl; and
each R is independently hydrogen or a protecting group (e.g t-butyl or benzyl).

Provided that when L is a group of the formula

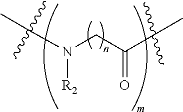

The combination of m and n result in a linear linker length of 3 to 21 atoms. For example, when m is 2 and each n is 4, the linker is 12 atoms in length. If m is 1, and n is 10, the linker length is also 12. Linker length is calculated using the formula m·(n+2).

As used herein, “protecting group” as used herein is a group introduced to a functional group (e.g., an phosphorous acid or carboxylic acid) that allows for chemoselectivity in a subsequent chemical transformation. Such groups, specifically carboxylic and phosphorus acid protecting groups, are described in Greene's Protective Groups in Organic Synthesis, 4th Edition (the relevant parts of which are incorporated by reference).

In some embodiments, a “protecting group” is alkyl, alkenyl, or haloalkyl. This includes, but is not limited to: methyl, ethyl, propyl, isopropyl, tert-butyl, allyl, trifluoromethyl or trifluoroethyl. In some embodiments, a “protecting group” is benzyl or substituted benzyl, which includes, but is not limited to, triphenylmethyl (trityl), diphenylmethyl, o-nitrobenzyl, 2,4,6-trimethylbenzyl, p-bromobenzyl, p-nitrobenzyl, p-methoxybenzyl (PMB), 2,6-dimethoxybenzyl, 4-(methylsulfinyl)benzyl, 4-sulfobenzyl, 4-azidomethoxybenzyl, and piperonyl.

In preferred embodiments, the radioligand imaging agent for use according to the present disclosure, is the PSMA-binding compound of formula (III):

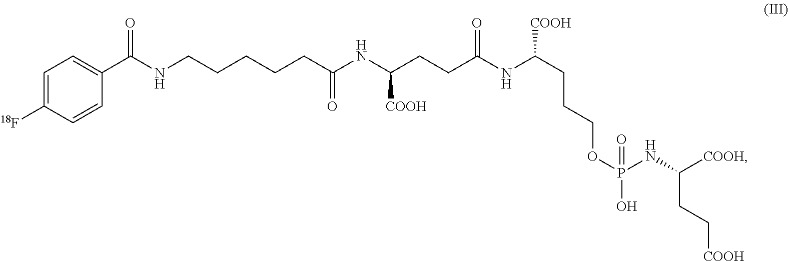 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof.

The compound of formula (III) is also referred in the literature as [¹⁸F]CTT1057.

Other PSMA-binding compounds for use according to the present disclosure and a method of synthesis of such compounds have been described in particular in WO2014/143736 which content is incorporated hereby in its entirety.

In specific embodiments, the PSMA-binding compound for use according to the present disclosure may be synthesized from the precursor CTT1298 of formula (IV) below,

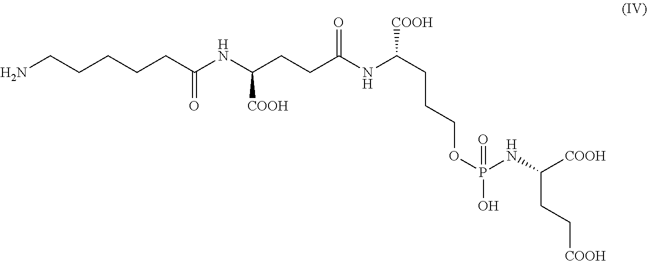 [SMILES: NCCCCCC(=O)N[C@@H](CCC(=O)N[C@@H](CCCOP(=O)(O)N[C@@H](CCC(=O)O)C(=O)O)C(=O)O)C(=O)O]

in particular, by coupling succinimidyl-¹⁸F-fluorobenzoate to the primary amine precursor as described in the following reaction scheme.

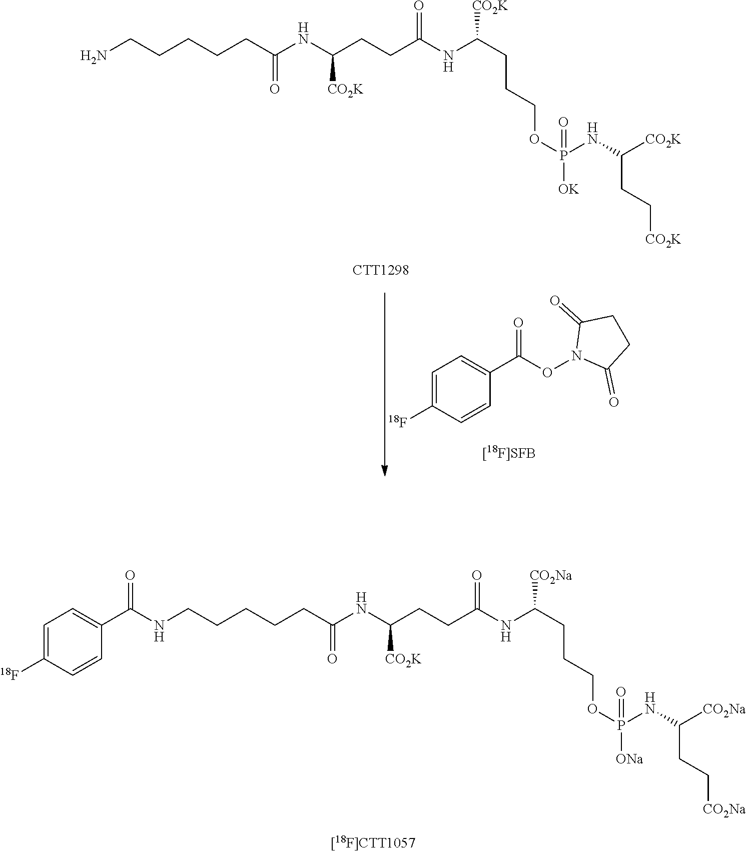

[¹⁸F]SFB may be synthesized through the following reaction scheme:

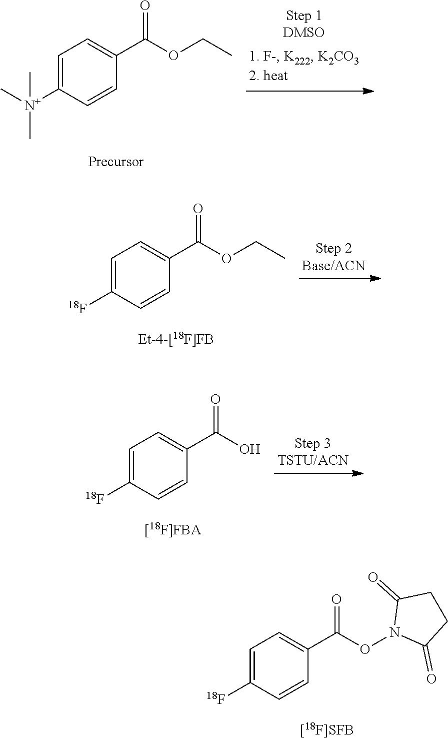

Methods for obtaining the compound CTT1298 have been described in the art and in particular in WO2014143736 (Example 1, which content is incorporated herein by reference).

Other examples of synthesis methods with ORA Neptis® Perform Synthesizer have further been described in Jivan et al 2017 (J Labelled Comp Radiopharm 2017: 60:1).

### The Pharmaceutical Composition of the Disclosure

The PSMA-binding compound for use according to the present disclosure is formulated as a pharmaceutical composition, typically a solution for injection or infusion.

Said solution for injection or infusion is preferably an aqueous or hydro-alcoholic solution which comprises the PSMA-binding compound as described herein, and one or more pharmaceutically acceptable excipients.

Typically, said PSMA-binding compound can be present in said pharmaceutical composition in a concentration providing a volumetric radioactivity between 150 and 1000 MBq/mL, preferably 200 and 700 MBq/mL, more preferably 250 and 450 MBq/mL, for example about 370 MBq/mL at calibration time.

The pharmaceutically acceptable excipient(s) can be any of those conventionally used. In particular, the one or more excipients can be selected from buffers, stabilizers against radiolytic degradation, isotonic agents, and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”. In preferred embodiment, a stabilizer against radiolytic degradation is ethanol.

Buffers include phosphate, acetate or citrate buffer or their combinations, preferably phosphate buffer. In specific embodiments, the buffer or combination of buffers is suitable for a pH between 6.5 and 7.5.

Isotonic agents include sodium chloride, in particular at a concentration of about 0.9%.

In specific embodiments, said solution for injection or infusion comprises the radioligand imaging agent as described in the previous section, for example, a PSMA-binding compound of formula (I), (II) or (III), preferably the compound of formula (III), at a concentration providing a volumetric radioactivity between 150 and 1000 MBq/mL, preferably 200 and 700 MBq/mL, more preferably 250 and 450 MBq/mL, at calibration time, typically about 370 MBq/mL at calibration time, and, optionally, a phosphate buffer and sodium chloride.

In specific embodiments, the solution for injection or infusion further comprises a buffer for a pH between 6.5 and 7.5, preferably phosphate buffer, and an isotonic agent, preferably sodium chloride.

In specific embodiments, the solution may further comprise a maximum amount of the precursor compound as used for synthesis. For example, the precursor compound of formula (IV) (also referred as CTT1298) is present in a concentration of not more than 5 μg/mL, preferably not more than 4 μg/mL, more preferably not more than 3 μg/mL, even more preferably not more than 2 μg/mL, even more preferably not more than 1 μg/mL.

In specific embodiments, the solution may further comprise a stabilizer against radiolysis that may be suitable as an eluent during the manufacturing of the solution, preferably said stabilizer and/or eluent is an alcohol, preferably ethanol. Indeed, according to a preferred embodiment, in particular with an automated synthesis of radioligand, for example as described in the Example below, the solution is obtained from the elution of the radioligand for separation from its precursor compound.

In a preferred embodiment, the solution for injection therefore comprises
(i) the radioligand imaging agent as described in the previous section, for example, a PSMA-binding compound of formula (I), (II) or (III), preferably the compound of formula (III), in a concentration providing a volumetric radioactivity between 250 and 450 MBq/mL at calibration time, typically at about 370 MBq/mL at calibration time,
(ii) NaCl in a concentration from 8.0 to 9.5 mg/mL; preferably from from 8.6 to 8.9 mg/mL,
(iii) NaH2PO4 in a concentration from 0.03 to 0.3 mg/mL; preferably from 0.1 to 0.2 mg/mL;
(iv) Na2HPO4 in a concentration from 0.2 to 1.2 mg/mL; preferably from 0.3 and 1.1 mg/mL;
(v) ethanol in a concentration from 5-50 mg/mL; preferably from 10.0 and 39.5 mg/mL; and,
(vi) optionally, a precursor compound (for example the CTT1298 precursor compound of formula IV) at a concentration of not more than 5.0 μg/mL, preferably not more than 4.0μg/mL, even more preferably not more than 3.0μg/mL, even more preferably not more than 2.0μg/mL, preferably not more than 1.0μg/mL.

### The Subject with Biochemical Recurrence

The detection methods and use of the radioligand imaging agent according to the present disclosure are intended for subjects with biochemical recurrence, preferably for subjects with prostate cancer with biochemical recurrence.

In specific embodiments, the detection methods and use of the radioligand imaging agent according to the present disclosure are intended for subjects with prostate cancer with biochemical recurrence after radical prostatectomy or after radiotherapy.

As used herein, the term “biochemical recurrence” refers to its general meaning as proposed by the American Urological Association criteria. More specifically, a definition of biochemical recurrence after radical prostatectomy is provided in Cookson et al 2007 J Urol; 177(2):540-5. In specific embodiments, it relates to a subject with prostate cancer having undergone radical prostatectomy and with detectable or rising of PSA level measured 6-13 weeks after surgery, that is superior or equal to 0.2 ng/ml, and optionally with a second confirmatory level, measured at least two weeks after the first measurement, which is strictly superior to 0.2 ng/ml. A definition of a subject with biochemical recurrence after radiotherapy may be provided by Roach et al 2006 Int J Radiat Oncol Biol Phys;65(4):965-74. In specific embodiments, it relates to a subject who have undergone curative intent radiotherapy with biochemical recurrence defined by American Society for Radiation Oncology (ASTRO)-Phoenix criteria (PSA superior or equal to 2 ng/ml above the nadir PSA, defined as the lowest PSA achieved (PSA nadir+2)).

### The Methods for Determining the Presence and Localization of Positive Tumors in a Subject with Biochemical Recurrence

An objective of the present disclosure is to provide methods for determining the presence or localization of PSMA-positive tumors in a subject with biochemical recurrence, typically in a subject with prostate cancer.

Typically, the presence and localization of PSMA-positive tumors is detected by analyzing the uptake of the PSMA-binding compound after injection of a radiotracer, for example, the PSMA-binding compound, in said subject which has been diagnosed with biochemical recurrence.

Accordingly, the disclosure relates to a method for determining the presence and/or the localization of PSMA positive tumors in a subject, wherein said subject has been diagnosed with biochemical recurrence, said method comprising
[i] administering to said subject, an effective dose of a radioligand imaging agent as described above, preferably a PSMA-binding compound of formula (I), (II) or (III), and most preferably a PSMA-binding compound of formula (III) below

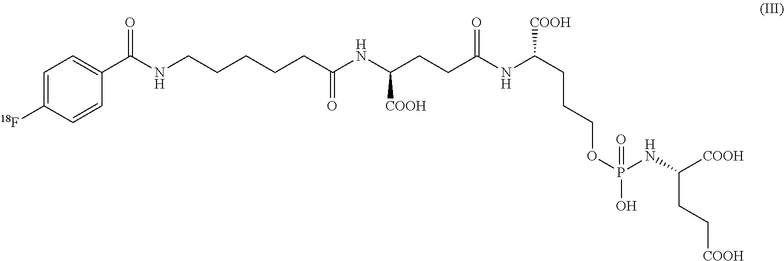 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof,
[ii] imaging said subject by PET scan, for example PET/CT or PET/MRI scan,
[iii] analysing the image obtained by the PET scan, thereby determining the presence and/or the localization of PSMA positive tumors in said subject.

Accordingly, the disclosure relates to a method for determining the presence and/or the localization of PSMA positive tumors in a subject with prostate cancer, wherein said subject has been diagnosed with biochemical recurrence, typically after radical prostatectomy or after radiotherapy, said method comprising
[i] administering to said subject, an effective dose of a radioligand imaging agent as described above, preferably a PSMA-binding compound of formula (I), (II) or (III), and most preferably a PSMA-binding compound of formula (III) below

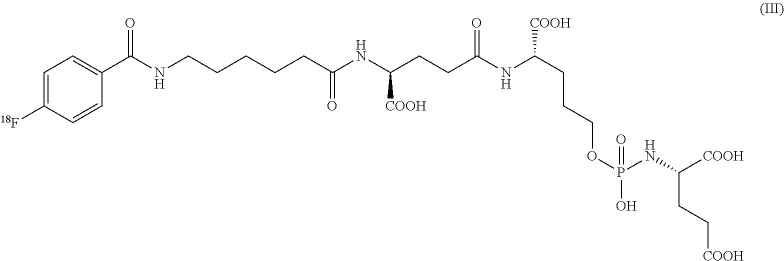 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof,
[ii] imaging said subject by PET scan, for example PET/CT or PET/MRI scan,
[iii] analysing the image obtained by the PET scan, thereby determining the presence and/or the localization of PSMA positive tumors in said subject.

In general, an effective dose is an amount of the imaging agent sufficient to yield an acceptable image using equipment which is available for clinical use. The amount of imaging agent used for the methods of the disclosure and the duration of the imaging step will depend upon, inter alia the body mass of the patient, the nature and severity of the condition to be detected, the nature of the therapeutic treatments which the patients has undergone, etc. Ultimately, the physician may decide the amount of the imaging agent to administer to each individual patient and the duration of the imaging step.

In specific embodiments, a subject diagnosed with biochemical recurrence receives a single effective dose of 250-450 MBq, typically about 370 MBq, by intravenous injection of a solution for injection or infusion comprising said radioligand imaging agent as described above. In specific embodiments, the volume of injection does not exceed 10 mL, for example is comprised between 500 μL and 10mL, preferably 800 μL μL and 5 mL, for example between 800 μL and 2 mL, and preferably about 1 mL.

Images of patient's body are then acquired by positron emission tomography—magnetic resonance imaging (PET/MRI) or positron emission tomography—computed tomography (PET/CT) imaging. Methods for acquisition of image by PET/MRI or PET/SCAN are well-known in the art.

Typically, the first PET scan is performed with a window from 60-120 minutes post-injection/infusion, for example at 90 minutes, with a possibility of a second PET scan performed up to 180 minutes after injection of the radioligand imaging agent.

The images are then analyzed, either by visual assessment, quantitative assessment or both, to identify the presence of one or more PSMA-positive lesions, and/or to determine the localization of one or more PSMA-positive lesions. Images can be generated by virtue of differences in the spatial distribution of the imaging agents which accumulate at a site when contacted with PSMA. The spatial distribution may be measured using any means, for example, the PET apparatus. The extent of accumulation of the imaging agent may be quantified using known methods for quantifying radioactive emissions. A particularly useful imaging approach may employ more than one imaging agent to perform simultaneous studies.

In specific embodiments, the term “PSMA-positive tumors” or “PSMA-positive lesions” refers to lesions visually identified in subject, preferably in subject with prostate cancer, to show pathological radioligand imaging agent uptake on PET/CT or PET/MRI as follows:
Visually PET positive lymph nodes considered greater than blood pool (adjacent or mediastinal blood pool);
PET positive bone lesions considered greater than physiologic bone marrow;
PET positive prostate, prostate bed and visceral lesions considered greater than physiologic background activity of the involvement organ or anatomic site, as previously described (Fendler WP, Calais J, Eiber M, et al (2019) JAMA Oncol; 5(6):856-63, Eiber M, Maurer T, Souvatzoglou M, et al (2015) J Nucl Med; 56(5):668-74, Ceci F, Uprimny C, Nilica B, et al (2015) Eur J Nucl Med Mol Imaging; 42:1284-94).

In certain embodiments of the method, the subject with biochemical recurrence has no PSMA-positive lesions detected by conventional imaging.

In certain embodiments of the method, the methods are expected to show improved sensitivity and/or specificity for detection of PSMA-positive tumor as compared to [⁶⁸Ga]-PSMA-11 compound, in particular in subject with prostate cancer with biochemical recurrence.

¹⁸F-labeled tracers have the following practical advantages: ¹⁸F has a longer half-life than ⁶⁸Ga, which enables the tracers to be distributed to PET centers without a cyclotron and to be easily handled in clinical routine. In addition, the higher positron decay branching of ¹⁸F (96.9%) versus ⁶⁸Ga (87.7%), together with the shorter positron range of ¹⁸F, account to the higher PET imaging resolution achieved with ¹⁸F-labeled radiopharmaceuticals (Conti M, Eriksson L (2016) EJNMMI Physics; 3(1): 1-17). Unlike most other PSMA agents labelled with either ⁶⁸Ga or ¹⁸F (e.g. [⁶⁸Ga]Ga-PSMA-11, [¹⁸F]PSMA1007, [¹⁸F]DCFPyL) which share a urea backbone, [¹⁸F]CTT1057 is based on a phosphoramidate scaffold that irreversibly binds to PSMA with high nanomolar affinity. This will lead to a higher resolution of the PET scans and to more precision and accuracy in detecting even very small lesions of the tumor.

In specific embodiments, the method is useful in particular in management of patients for treating the recurrence.

Hence, the present disclosure also relates to a method for monitoring the disease state of a subject with biochemical recurrence, said method comprising
[i] administering to said subject, an effective dose of a radioligand imaging agent as described above, for example, a PSMA-binding compound of formula (I), (II) or (III) or any pharmaceutically acceptable salts thereof, and more preferably a PSMA-binding compound of formula (III) below

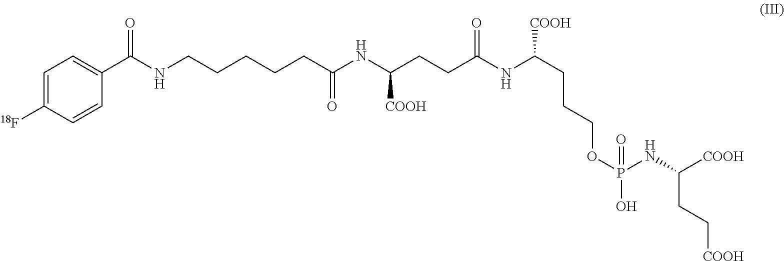 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof, preferably by intravenous injection of a solution of injection as described previously,
[ii] imaging said subject by a first positron emission tomography (PET) scan, for example with a window from 60-120 minutes post-injection, preferably at about 90 minutes, and optionally, a second PET scan, up to 180 minutes post injection,
[iii] analysing the image obtained by the PET scan,
[iv] determining the presence and/or the localization of PSMA positive tumors in said subject,
[v] determining a treatment option depending on the presence and/or localization of PSMA positive tumors in said subject,
[vi] optionally treating the subject with said treatment option.

Correct identification of disease location and extent determines treatment decisions for patients, typically patients with prostate cancer. Identification of distant metastatic disease at the early stages of prostate cancer is important in planning prostate cancer management. There is increasing evidence that primary landing sites for prostate cancer lie outside the template of an extended pelvic lymph node dissection (ePLND). Primary lymph node landing sites outside an ePLND have been reported in 47.7% of men with suspected node-positive disease on ⁶⁸Ga-PSMA PET/CT (Yaxley JW, Raveenthiran S, Nouhaud FX, et al (2019a) BJU Int; 124:401-7). This is very important, as the morbidity of a surgery could be avoided, given the change in management from one of curative intent, to management that will require a multimodality approach after treatment of the prostate primary tumor (Yaxley JW, Dagher J, Delahunt B, et al (2018) World J Urol; 36:15-20, Yaxley JW, Raveenthiran S, Nouhaud FX, et al (2019b) J Urol; 201:815-20).

Hence, treatment options may subsequently change among different approaches such as Surgery, Radiation alone, Radiation plus androgenic deprivation therapy, androgenic deprivation therapy alone, Observation/surveillance, or other.

The disclosure further relates to a kit for monitoring the disease state of a subject as described above, said kit comprising at least an effective dose of a radioligand imaging agent as described above, for example a PSMA-binding compound of formula (I), (II) or (III) and more preferably a PSMA binding compound of formula (III) below

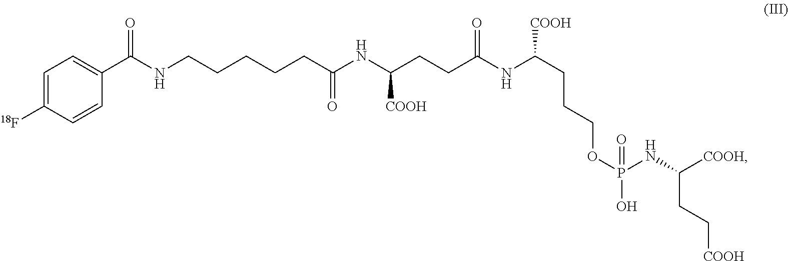 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any of its pharmaceutically acceptable salts thereof,
or a solution for injection or infusion comprising said radioligand imaging agent as described previously.

The disclosure further relates to kit for use of the methods as above described, said kit comprising an effective dose, for example about 370 MBq of the radioligand imaging agent or their precursors for the synthesis as described above, in combination with a pharmaceutically acceptable carrier. The imaging agent, their precursors and the carrier are provided in solution

In certain embodiments, a kit for use in the methods of the present disclosure comprises a non-radiolabeled precursor, typically the compound of formula (IV), to be combined with a radiolabeled reagent on-site, such as K[¹⁸F] or Na[¹⁸F].

### In Accordance with the Present Disclosure the Following Embodiments E1 to E30 are Provided

E1: A radioligand imaging agent, for use in a diagnostic method for determining the presence and/or localization of PSMA-positive tumors in a subject, particularly said subject is a subject with prostate cancer, and said PSMA-positive tumors is prostate cancer, wherein said subject has been diagnosed with biochemical recurrence, and wherein said radioligand imaging agent is a PSMA-binding compound comprising a phosphoramidate group and a [¹⁸F]-fluoro group.

E1b: The radioligand imaging agent for use according to E1, said method comprising
[i] administering to said subject, an effective dose of said radioligand imaging agent,
[ii] imaging said subject by PET scan, either PET/CT or PET/MRI scan,
[iii] analysing the image obtained by the PET scan, and,
[iv] determining the presence and/or the localization of PSMA positive tumors in said subject.

E2: The radioligand imaging agent for use according to E1 or E1b, where said agent is a PSMA-binding compound of formula (I):

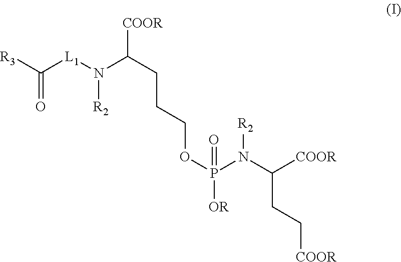

or any pharmaceutically acceptable salts thereof, wherein
each R is independently hydrogen or a protecting group (e.g t-butyl or benzyl).
each R2 is independently hydrogen or C1-C6 alkyl,
R3 is a phenyl or pyridyl, each substituted with [¹⁸F]-fluoro group and optionally substituted with a second group selected from the group consisting of halo, cyano and nitro,
and L1 is a linker, preferably comprising one or more groups selected from one or more amino-acids, C1-C18 alkylene, and heteroalkylene comprising 1 to 35 carbon atoms and 1 to 15 heteroatoms, said heteroalkylene being optionally substituted by one or more substituents selected from oxo and C1-C6 alkyl, and more preferably L1 is a linker selected from 1 to 6 amino-acids.

E3: The radioligand imaging agent for use according to any one of E1, E1b or E2, where said radioligand imaging agent is a PSMA-binding compound of formula (II):

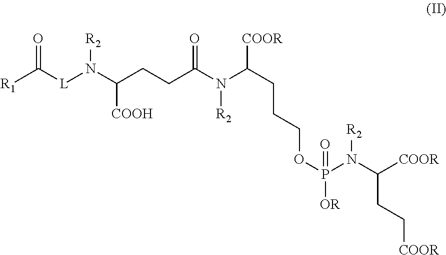

and any pharmaceutically acceptable salts thereof, wherein
L is a linker comprising a moiety of the formula —NH—CH₂CH₂—(OCH₂CH₂—)y—C(O)— or a group of the formula

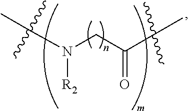

wherein y is 1, 2, 3, 4, 5, 6, 7, 8, 9, 10, 11, or 12:
m is 1, 2, 3, or 4;
each n is independently 1, 2, 3, 4, 5, 6, 7, 8, 9, 10, 11, or 12;
R1 is a phenyl or pyridyl, each substituted with [¹⁸F]-fluoro group and optionally substituted with a second group selected from the group consisting of halo, cyano and nitro;
each R2 is independently hydrogen or C1-C6 alkyl; and
each R is independently hydrogen or a protecting group (e.g t-butyl or benzyl)

Provided that when L is a group of the formula

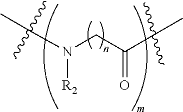

the combination of m and n result in a linear linker length of 3 to 21 atoms.

E4: The radioligand imaging agent for use according to any one of E1-E3, wherein said radioligand imaging agent is the PSMA-binding compound of formula (III):

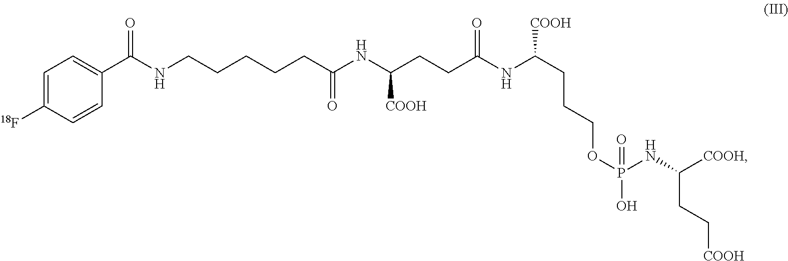 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof.

E5: The radioligand imaging agent for use according to any one of E1-E4, wherein said subject has been diagnosed with biochemical recurrence after radical prostatectomy or radiotherapy.

E6: The radioligand imaging agent for use according to any one of E1-E5, wherein said radioligand imaging agent is formulated as a solution for injection or infusion in a concentration providing a volumetric radioactivity from 150 to 1000 MBq/mL, for example 370 MBq/mL+/−10% at calibration time.

E7: The radioligand imaging agent for use according to any one of E1-E6, wherein said radioligand imaging agent is administered intravenously at an effective dose comprised between 250 and 450 MBq, typically about 370 MBq.

E8: The radioligand imaging agent of E6 or E7, wherein a first PET scan imaging of the subject is performed between 60 and 120 minutes post-injection or infusion, and optionally a second scan imaging is performed up to 180 minutes post-injection or infusion.

E9: A solution for injection or infusion, which is an aqueous solution comprising said radioligand imaging agent as defined in any of E1-E5, in a concentration providing a volumetric radioactivity between 150 and 1000 MBq/mL, for example about 370 MBq/mL, and one or more pharmaceutically acceptable excipients.

E10: The solution of Claim E9, in which the precursor compound of formula (IV)

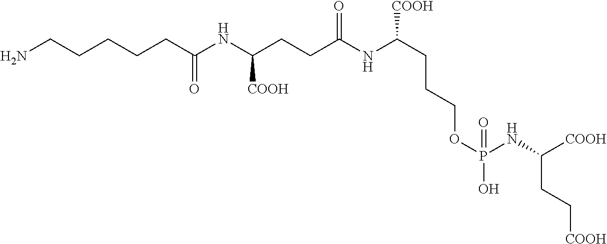 [SMILES: NCCCCCC(=O)N[C@@H](CCC(=O)N[C@@H](CCCOP(=O)(O)N[C@@H](CCC(=O)O)C(=O)O)C(=O)O)C(=O)O]

is present in a concentration of not more than 5.0 μg/mL, preferably not more than 4.0 μg/mL, more preferably not more than 3.0 μg/mL, even more preferably not more than 2.0 μg/mL, even more preferably not more than 1.0 μg/mL.

E11: The solution of E9 or E10, further comprising a buffer for a pH between 5.0 and 8.0, preferably between 6.0 and 8.0, more preferably between 6.5 and 7.5, preferably phosphate buffer, and an isotonic agent, preferably sodium chloride.

E12: The solution of E11, further comprising a stabilizer against radiolysis, preferably a stabilizer that is suitable as an eluent during the manufacturing of the solution, preferably said stabilizer and/or eluent is an alcohol, preferably ethanol.

E13: The solution of E12, comprising
[i] NaCl in a concentration from 8.0 to 9.5 mg/mL; preferably from 8.6 to 8.9 mg/mL,
[ii] NaH₂PO₄ in a concentration from 0.03 to 0.3 mg/mL; preferably from 0.1 to 0.2 mg/mL;
[iii] Na₂HPO₄ in a concentration from 0.2 to 1.2 mg/mL; preferably from 0.3 and 1.1 mg/mL;
[iv] ethanol in a concentration from 5-50 mg/mL; preferably from 10.0 and 39.5 mg/mL; and,
[v] optionally, a precursor compound at a concentration less than 5.0 μg/mL.

E14: A method for determining the presence and/or the localization of PSMA positive tumors in a subject, preferably a subject with prostate cancer, wherein said subject has been diagnosed with biochemical recurrence, said method comprising
[i] administering to said subject, an effective dose of a radioligand imaging agent as defined in any of embodiments E1-E7,
[ii] imaging said subject by PET scan, either PET/CT or PET/MRI scan,
[iii] analysing the image obtained by the PET scan,
thereby determining the presence and/or the localization of PSMA positive tumors in said subject.

E15: The method of E14, wherein said radioligand imaging agent is administered intravenously at an effective dose comprised between 250 and 450 MBq, typically about 370 MBq.

E16: The method of E14 or E15, which determines the presence and/or the localization of PSMA positive tumor lesions of a size from 5 to 10 mm.

E17: The method of E14-E16, wherein the step (ii) of imaging comprises a first PET scan performed from 60 to 120 minutes post injection/infusion, typically about 90 minutes post-injection/infusion, and optionally a second PET scan is performed up to 180 minutes post-injection/infusion.

E18: The method of any one of E14-E17, wherein said radioligand imaging agent is formulated as a solution for injection or infusion as defined in any one of embodiments E9-E13.

E19: A method for monitoring the disease state of a subject with biochemical recurrence, said method comprising
[i] administering to said subject, an effective dose of a radioligand imaging agent as described above, for example, a PSMA-binding compound of formula (I), (II) or (III) or any pharmaceutically acceptable salts thereof, and more preferably a PSMA-binding compound of formula (III) below

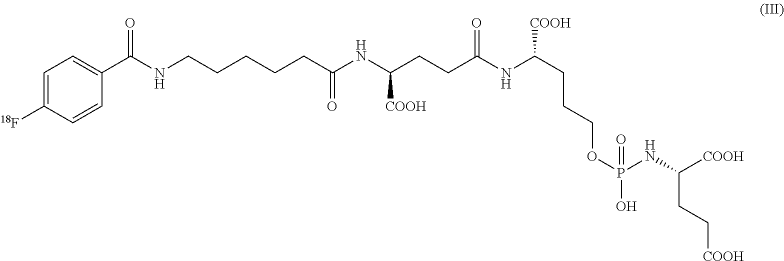 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof, preferably by intravenous injection of a solution of injection as described previously,
[ii] imaging said subject by a first positron emission tomography (PET) scan, for example with a window from 60-120 minutes, preferably at about 90 minutes, and optionally, a second PET scan, up to 180 minutes post injection,
[iii] analysing the image obtained by the PET scan,
[iv] determining the presence and/or the localization of PSMA positive tumors in said subject,
[v] determining a treatment option depending on the presence and/or localization of PSMA positive tumors in said subject, and,
[vi] optionally treating the subject with said treatment option.

E20. The method of E19, wherein said subject is with prostate cancer.

E21. The method of E20, wherein said subject has been diagnosed with biochemical recurrence after radical prostatectomy or radiotherapy.

E22. A process for manufacturing a radioligand imaging agent for use in a diagnostic method comprising the steps as defined in any one of E14-18, wherein said radioligand imaging agent is as defined in any of E1-E9.

E23: A solution for injection or infusion, which is an aqueous solution comprising a radioligand imaging agent being a PSMA-binding compound comprising a phosphoramidate group and a [¹⁸F]-fluoro group, in a concentration providing a volumetric radioactivity between 150 and 1000 MBq/mL, for example about 370 MBq/mL, and one or more pharmaceutically acceptable excipients.

E24: The solution of E23, wherein said radioligand imaging agent is a PSMA-binding compound of formula (I):

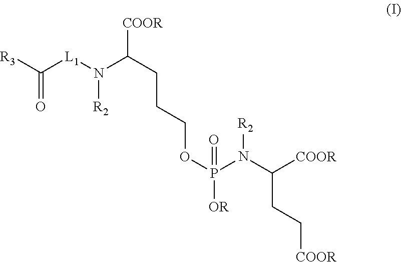

or any pharmaceutically acceptable salts thereof, wherein
each R is independently hydrogen or a protecting group (e.g t-butyl or benzyl).
each R2 is independently hydrogen or C1-C6 alkyl,
R3 is a phenyl or pyridyl, each substituted with [¹⁸F]-fluoro group and optionally substituted with a second group selected from the group consisting of halo, cyano and nitro,
and L1 is a linker, preferably comprising one or more groups selected from one or more amino-acids, C1-C18 alkylene, and heteroalkylene comprising 1 to 35 carbon atoms and 1 to 15 heteroatoms, said heteroalkylene being optionally substituted by one or more substituents selected from oxo and C1-C6 alkyl, and more preferably L1 is a linker selected from 1 to 6 amino-acids.

E25: The solution according to any one of E23 or E24, wherein said radioligand imaging agent is a PSMA-binding compound of formula (II):

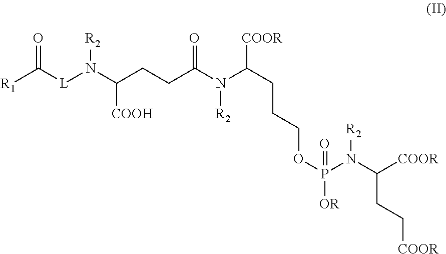

and any pharmaceutically acceptable salts thereof, wherein
L is a linker comprising a moiety of the formula —NH—CH₂CH₂—(OCH₂CH₂—)y—C(O)— or a group of the formula

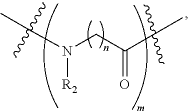

wherein y is 1, 2, 3, 4, 5, 6, 7, 8, 9, 10, 11, or 12:
m is 1, 2, 3, or 4;
each n is independently 1, 2, 3, 4, 5, 6, 7, 8, 9, 10, 11, or 12;
R1 is a phenyl or pyridyl, each substituted with [¹⁸F]-fluoro group and optionally substituted with a second group selected from the group consisting of halo, cyano and nitro;
each R2 is independently hydrogen or C1-C6 alkyl; and
each R is independently hydrogen or a protecting group (e.g t-butyl or benzyl)

Provided that when L is a group of the formula

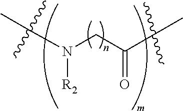

the combination of m and n result in a linear linker length of 3 to 21 atoms.

E26: The solution according to any one of E23-E25, wherein said radioligand imaging agent is the PSMA-binding compound of formula (III):

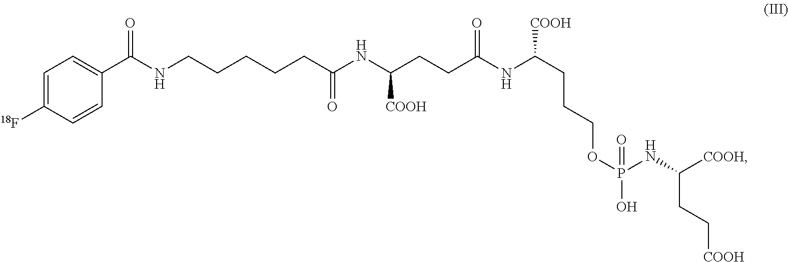 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]

or any pharmaceutically acceptable salts thereof.

E27: The solution according to any one of E23-E26, in which a precursor compound of formula (IV)

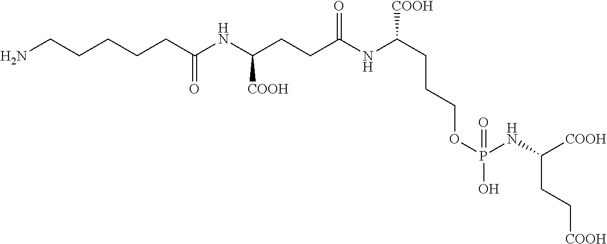 [SMILES: NCCCCCC(=O)N[C@@H](CCC(=O)N[C@@H](CCCOP(=O)(O)N[C@@H](CCC(=O)O)C(=O)O)C(=O)O)C(=O)O]

is present in a concentration of not more than 5.0 μg/mL, preferably not more than 4.0 μg/mL, more preferably not more than 3.0 μg/mL, even more preferably not more than 2.0 μg/mL, even more preferably not more than 1.0 μg/mL.

E28: The solution according to any one of E23-E27, further comprising a buffer for a pH between 5.0 and 8.0, preferably between 6.0 and 8.0, more preferably between 6.5 and 7.5, preferably phosphate buffer, and an isotonic agent, preferably sodium chloride.

E29: The solution according to any one of E23-E28, further comprising a stabilizer against radiolysis, preferably a stabilizer that is suitable as an eluent during the manufacturing of the solution, preferably said stabilizer and/or eluent is an alcohol, preferably ethanol.

E30: The solution of E29, comprising
[i] NaCl in a concentration from 8.0 to 9.5 mg/mL; preferably from 8.6 to 8.9 mg/mL,
[ii] NaH₂PO₄ in a concentration from 0.03 to 0.3 mg/mL; preferably from 0.1 to 0.2 mg/mL;
[iii] Na₂HPO₄ in a concentration from 0.2 to 1.2 mg/mL; preferably from 0.3 and 1.1 mg/mL;
[iv] ethanol in a concentration from 5-50 mg/mL; preferably from 10.0 and 39.5 mg/mL; and,
[v] a precursor compound at a concentration less than 5.0 μg/mL.

### EXAMPLES

### Example 1: Manufacturing of a Solution Comprising the Radioligand Imaging Agent

The Drug Product is a diluted solution of the [¹⁸F]CTT1057 concentrated mother solution (radioactive drug substance, 15±1 mL, with about 1685-6667 MBq/mL at Tm) in NaCl 0.9% to adjust the volumic activity of the final solution at 370 MBq/mL±10% (Tc). The volume of saline added is calculated according to the activity of [¹⁸F]CTT1057 obtained at the end of the synthesis (Tm), decay corrected at the time of calibration (Tc). Tm is the time of measurement of the activity in the mother solution. Tm is some minutes after the EOS.

The final volume of drug product ranges between 15-59 mL, and the quantitative composition of [¹⁸F]CTT1057 finished product varies accordingly.

The qualitative and quantitative composition of the drug product in the nominal volumes of 15 mL and 59 mL are described in Table 1.

TABLE 1
Qualitative and Quantitative composition of 1 mL of drug product

| | | Quantity per batch | | Quantity per batch | |
|---|---|---|---|---|---|
| Components | Role | (V_{T} = 15 mL**) | | (V_{T} = 59 mL**) | |
| [¹⁸F]CTT1057 | Active substance | 370 MBq at Tc | | 370 MBq at Tc | |
| CTT1298 | Residual Chemical | ≤5.0 · 10⁻³ | mg* | ≤5.0 · 10⁻³ | mg* |
| | precursor | | | | |
| NaCl | Isotonic agent | 8.55 | mg | 8.89 | mg |
| NaH₂PO₄ | Buffer | 0.24 | mg | 0.06 | mg |
| Na₂HPO₄ | Buffer | 1.07 | mg | 0.27 | mg |
| EtOH | Eluent, stabilizer | 39.45 | mg | 10.03 | mg |

| | | | |
|---|---|---|---|
| Water for injection | Solvent | q.s. 1 mL | q.s. 1 mL |
| (WFI) | | | |

Notes:
Tc = Time of calibration
V_{T} = Total volume
*Considering the maximum injected dose of 10 mL
**This amount includes water for injection present in the 25 mL vial (0.20 ± 0.02 mL) and in the primary packaging vial 15 ml (0.10 ± 0.01 mL), which is introduced during the sterilization process in amounts that are considered negligible.
The synthesis of the drug substance ([¹⁸F]CTT1057) and its formulation into the drug product ([¹⁸F]CTT1057 370 MBq/mL solution for injection), are part of an automated continuous process as described in the next sections.

### Synthesis of the Drug Substance

### [¹⁸F]CTT1057 Active Substance (Mother Solution) is Obtained in a Synthetic Route of two Stages

In the first stage (Step 1), [₁₈F]SFB prosthetic group is prepared in a one-pot, three-step procedure, starting with a radiofluorination of the FB starting material, followed by a saponification of the ethyl esther and a coupling with TSTU.

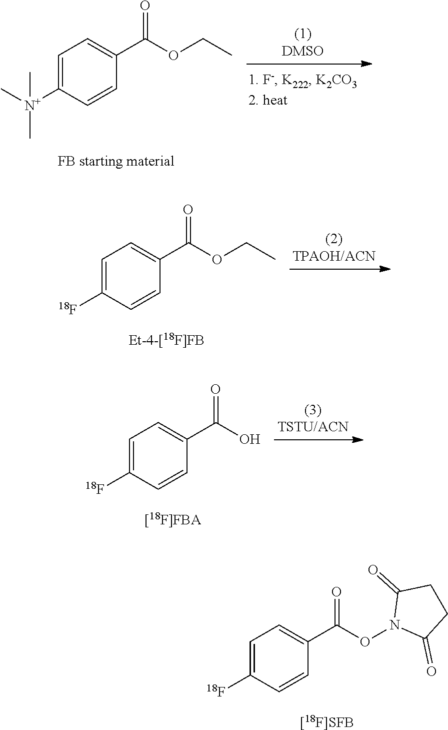

In the second stage (Step 2), the labelling of the precursor CTT1298 with the isolated [18F]SFB in basic mild conditions leads to the formation of [¹⁸F]CTT1057

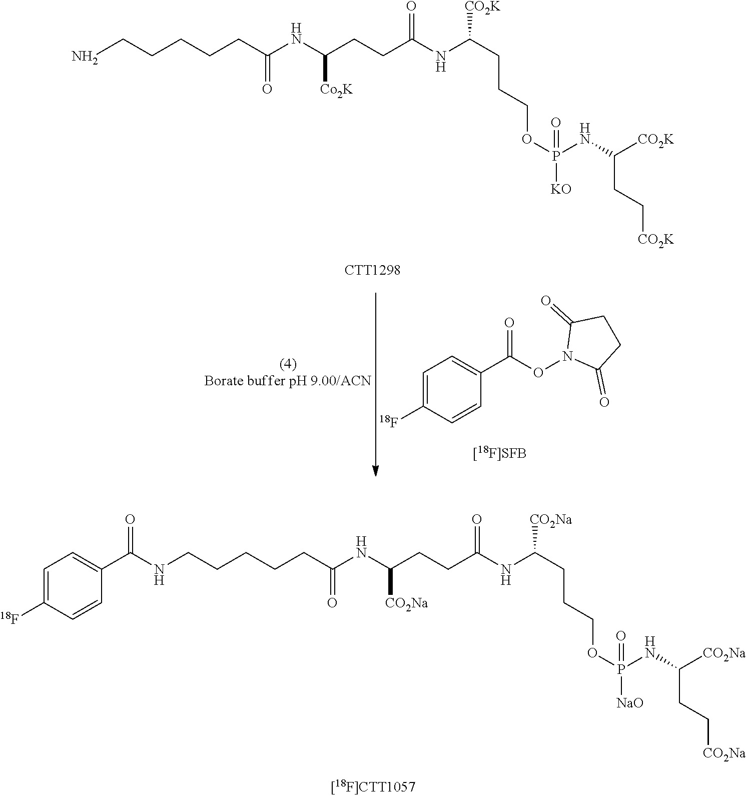

A two-step purification process drives to the separation of by-products and residual reagents and the final formulated [¹⁸F]CTT1057, of a radiochemical purity ≥95%, is obtained.

The entire synthesis and purification is automatically conducted in a synthesizer as described hereafter.

### Manufacturing Process of the Mother Solution Containing the Drug Substance

Production of Radionuclidic Precursor ([¹⁸F]fluoride) from [¹⁸O]H₂O

The radionuclide is obtained in the form of [¹⁸F]fluoride ions by bombardment of ≥97% pure [¹⁸O]water with an intensive beam of accelerated protons. This nuclear reaction is produced in a cyclotron target.

### Transfer of Radioactivity and ¹⁸F Recovery in the Clean Room

After bombardment, the irradiated [¹⁸O]water containing the [¹⁸F]fluoride is automatically transferred into a dedicated synthesis module.

Production of [¹⁸F]CTT1057 from [¹⁸F]fluoride

Production of [¹⁸F]CTT1057 from [¹⁸F]fluoride takes place inside a lead shielded isolator in two steps described below.

### Manufacture of [¹⁸F]SFB Prosthetic Group

[¹⁸F]SFB prosthetic group is prepared in a one-pot, three-step procedure, comprising the radiofluorination of the FB precursor starting material to Et-4-[18F]FB, then the saponification of the ethyl esther to obtain [¹⁸F]FBA and the coupling of [¹⁸F]FBA with TSTU (N,N,N′,N′-Tetramethyl-O-(N-succinimidyl)uronium tetrafluoroborate) to [¹⁸F]SFB.

[¹⁸F]SFB (N-succinimidyl-4-[18F]fluorobenzoate) is then purified through an HLB purification cartridge. [¹⁸F]SFB is retained in the cartridge while non-reacted [¹⁸F]fluoride is moved to the waste.

Finally, pure [¹⁸F]SFB is eluted from the HLB with acetonitrile into a second reactor containing the precursor CTT1298 in solution.

### Manufacture of [18F]CTT1057 Drug Substance

[¹⁸F]CTT1057 is obtained by a reaction between the prosthetic group [¹⁸F]SFB and the precursor CTT1298 including:

Coupling to CTT1298—[¹⁸F]SFB is eluted from the HLB, in the second reactor, containing CTT1298 solution. The reaction is carried out in the second reactor at 40° C. during 12 min.

CTT1057 purification—the crude is diluted in water and two-step purification process drives to the separation of by-products and residual reagents. Diluted crude is passed through a QMA cartridge towards the waste. [¹⁸F]CTT1057 is retained in the QMA, together with other undesired species. The QMA is rinsed with a solution 0.09% NaCl/20% EtOH to waste, and [¹⁸F]CTT1057 eluted with 20 mM phosphate buffer at pH 2.0 and trapped again onto the HLB in a time-controlled step to minimize the decomposition of [¹⁸F]CTT1057 in acidic media.

CTT1057 formulation—The pure, final formulated [¹⁸F]CTT1057 is eluted, neutralized and formulated from the HLB in one single step by a 5% ethanol in pH 7.4 phosphate buffered saline solution formulation solution.

### Manufacturing Process Development

The drug substance [¹⁸F]CTT1057 was developed in a one-step reaction between the prosthetic group [¹⁸F]SFB and the chemical precursor CTT1298. To ensure an efficient reaction conversion, non-radioactive [¹⁹F]SFB and CTT1298 were used and several coupling reaction conditions were tested: different buffered media over a range of pH from 7 to 11, temperatures from ≈25° C. to 60° C., and duration from 5 to 10 minutes.

The final adjustment of the time of reaction was optimized using [¹⁸F]CTT1057 productions to have the real concentrations condition and to take into account the reaction time versus decay balance.

The final purification step of [¹⁸F]CTT1057 on SPE cartridge(s) was key to deliver a product with adequate purity. The reaction crude in basic media was diluted and first purified with a QMA (Quaternary Methyl Ammonium) cartridge that allowed removal of most of the radiochemical impurities.

However, the elution of the purified [¹⁸F]CTT1057 with physiological saline evidenced the presence of the chemical precursor CTT1298 in high amounts.

The separation of CTT1298 from [¹⁸F]CTT1057 was therefore challenging due to the similarity of the two molecules. The final strategy focused on the small difference of polarity caused by [¹⁸F]CTT1057 aromatic ring, which allowed a selective retention of the final product using a HLB cartridge. The final elution and formulation of [¹⁸F]CTT1057 drug substance was optimised to one-step using phosphate buffered saline solution containing 5% ethanol.

### Preparation of the Solution for Injection

The Drug Product is a diluted solution of the [¹⁸F]CTT1057 concentrated mother solution (radioactive drug substance, 15±1 mL) in NaCl 0.9% to adjust the volumic activity of the final solution at 370 MBq/mL±10% (Tc). The volume of saline added is calculated according to the activity of [¹⁸F]CTT1057 obtained at the end of the synthesis (Tm), decay corrected at the time of calibration (Tc). The final volume of drug product ranges between 15-59 mL, and the quantitative composition of [¹⁸F]CTT1057 finished product varies accordingly.

The qualitative and quantitative composition of the drug product in the nominal volumes of 15 mL and 59 mL are described in Table 2.

TABLE 2
Qualitative and Quantitative composition of 1 mL of drug product

| | | Quantity per batch | | Quantity per batch | |
|---|---|---|---|---|---|
| Components | Role | (VT = 15 mL**) | | (VT = 59 mL**) | |
| [¹⁸F]CTT1057 | Active substance | 370 MBq at Tc | | 370 MBq at Tc | |
| CTT1298 | Chemical precursor | ≤5.0 · 10⁻³ | mg* | ≤5.0 · 10⁻³ | mg* |
| NaCl | Isotonic agent | 8.55 | mg | 8.89 | mg |
| NaH2PO4 | Buffer | 0.24 | mg | 0.06 | mg |
| Na2HPO4 | Buffer | 1.07 | mg | 0.27 | mg |
| EtOH | Eluent, Stabilizer | 39.45 | mg | 10.03 | mg |

| | | | |
|---|---|---|---|
| | against radiolysis | | |
| Water for injection | Solvent | q.s. 1 mL | q.s. 1 mL |
| (WFI) | | | |

Notes:
Tc = Time of calibration
VT = Total volume
*Considering the maximum injected dose of 10 mL
**This amount includes water for injection present in the 25 ml vial (0.20 ± 0.02 mL) and in the primary packaging vial 15 ml (0.10 ± 0.01 mL), which is introduced during the sterilization process in amounts that are considered negligible.

The synthesis of the drug substance ([¹⁸F]CTT1057) and its formulation into the drug product ([¹⁸F]CTT1057 370 MBq/mL solution for injection) are part of an automated continuous process comprising the steps below:

Reception in the dispensing cell—Final formulated [¹⁸F]CTT1057 bulk concentrated mother solution is transferred the synthesis cell to the dispensing cell.

### Measurement of the Weight and Radioactivity of Mother Solution

The 25 mL vial is weighed. The net weight of [¹⁸F]CTT1057 bulk concentrated mother solution is defined by the difference between the weight of the product vial and the empty vial. The activity contained in the vial is measured with dose calibrator.

Transfer of the ¹⁸F-CTT1057 solution—[¹⁸F]CTT1057 bulk concentrated mother solution is transferred from the 25 ml vial into an empty, sterile Mother Vial.

Dilution—The Mother Vial with [¹⁸F]CTT1057 bulk concentrated mother solution is placed on the balance. The designated amount of NaCl 0.9% is transferred into the Mother Vial, automatically until the right amount of NaCl is added. The [¹⁸F]CTT1057 diluted mother solution with a concentration of 370 MBq/ml±10% at calibration time Tc (Tc=T0+4 h) is obtained.

Homogenization of the final ¹⁸F-CTT1057 solution (mixing)—The final [¹⁸F]CTT1057 diluted mother solution is mixed.

Final Filtration and Distribution in the final 15 ml vials—[¹⁸F]CTT1057 diluted mother solution is distributed using semiautomatic dispensing system.

The final [¹⁸F]CTT1057 diluted mother solution in the vial is connected to the filter (used for final filtration) and a sterile needle fixed to the dispensing automaton, through the pump via a sterile tube.

### Example 2: Clinical Study

### Protocol Summary

| | |
|---|---|
| Full Title | Phase III study for evaluation of the diagnostic performance of [¹⁸F]CTT1057 |
| | PET imaging in patients with prostate cancer with rising PSA levels |
| | [biochemical recurrence (BCR)] |
| Brief title | Study of diagnostic performance of [¹⁸F]CTT1057 in BCR |
| Investigation | Radiopharmaceutical |
| type | |
| Purpose and | [¹⁸F]CTT1057 is a promising novel Prostate Specific Membrane Antigen |
| rationale | (PSMA)-targeting ¹⁸F-labeled Positron Emission Tomography (PET) |
| | imaging agent. Unlike other PSMA agents which share a urea backbone, |
| | [¹⁸F]CTT1057 is based on a phosphoramidate scaffold that irreversibly binds |
| | to PSMA with high nanomolar affinity, which may account for a higher and |
| | prolonged tumour uptake. Together with the higher resolution of |
| | PET/Computerized Tomography (CT) images when using a ¹⁸F-labelled PET |
| | agent, it would therefore favor the identification of smaller lesions, and |
| | consequently, also at earlier stages of the disease. A [¹⁸F]CTT1057 Phase-I |
| | study has shown an acceptable safety profile without any radiotracer-related |
| | adverse reactions, and has provided preliminary evidence of diagnostic |
| | performance of [¹⁸F]CTT1057 PET in detecting and localizing PSMA- |
| | positive tumors using pathology as standard of truth (SoT). |
| | The current study aims at evaluating the diagnostic performance of |
| | [¹⁸F]CTT1057 as a PET imaging agent for detection and localization of |
| | PSMA positivity in patients diagnosed of biochemical recurrence of prostate |
| | cancer (Prostate Cancer), using a composite truth standard. |
| Primary | The co-primary objectives of this study are |
| Objective(s) | to evaluate the region-level correct localization rate (CLR) of |
| | [¹⁸F]CTT1057 |
| | to evaluate the patient-level positive predictive value (with anatomical |
| | localization) of [¹⁸F]CTT1057 |
| | The primary clinical question of interest is: What is the probability that a |
| | positive [¹⁸F]CTT1057 PET/CT scan truly detects and localizes (PSMA- |
| | expressing) tumor recurrence in BCR prostate cancer patients? |
| Secondary | To evaluate the patient-level sensitivity of [¹⁸F]CTT1057 |
| Objectives | To evaluate the patient-level specificity of [¹⁸F]CTT1057 |
| | To evaluate the patient-level negative predictive value of [¹⁸F]CTT1057 |
| | To evaluate the patient-level accuracy of [¹⁸F]CTT1057 |
| | To evaluate the region level sensitivity of [¹⁸F]CTT1057 |
| | To evaluate the region level specificity of [¹⁸F]CTT1057 |
| | To evaluate the region level negative predictive value of [¹⁸F]CTT1057 |
| | To evaluate the region level accuracy of [¹⁸F]CTT1057 |
| | To evaluate correct detection rate (CDR) |
| | To evaluate detection rate |
| | To evaluate the patient-level positive predictive value related to PSA |
| | levels |
| | To characterize the safety and tolerability of [¹⁸F]CTT1057 |
| | [¹⁸F]CTT1057 scan inter-reader variability |
| | [¹⁸F]CTT1057 scan intra-reader variability |
| | Concordance between [¹⁸F]CTT1057 and [⁶⁸Ga]Ga-PSMA-11 for |
| | detection of lesions at patient level |
| | To evaluate the change in patient management plans attributed to the |
| | PET/CT scan |
| | To assess all the above primary and secondary objectives independently |
| | in the subgroup of patients post-prostatectomy and the subgroup of |
| | patients after prostate radical Radiotherapy |
| Study design | This is a prospective, open-label, multi center, single-arm Phase III study to |
| | evaluate the diagnostic performance of [¹⁸F]CTT1057 as a PET imaging |
| | agent for detection and localization of PSMA positive tumors in prostate |
| | cancer patients diagnosed with biochemical recurrence (BCR) after radical |
| | prostatectomy, using a composite truth standard (CTS) as reference. |
| | The CTS to be used as reference will be hierarchical in nature, with 3 levels |
| | of Standard of Truth (SoT) procedures, that will be applied as follows: |
| | CTS Level 1: Histopathology if available (from prospective biopsy or |
| | salvage surgery performed within 8 weeks after the [¹⁸F]CTT1057 PET/CT |
| | scan); OR in case that histopathology is not available, inconclusive or |
| | negative: |
| | CTS Level 2: Imaging diagnostic procedures performed on each patient as |
| | clinically indicated per SoC, which must include at least a high resolution CT |
| | scan with contrast and a [⁶⁸Ga]Ga-PSMA-11 PET/CT) performed within 8 |
| | weeks (either before or after) the [¹⁸F]CTT1057 PET/CT scan. Three-month |
| | follow-up imaging (from baseline) will also be used as part of the CTS level |
| | 2 in cases where it is clinically required for the diagnosis of particular |
| | lesion(s); OR if neither of the two above are feasible or deemed appropriate: |
| | CTS Level 3: 50% or greater decline in PSA following radiation |
| | therapy (as long as no concomitant androgen deprivation therapy (ADT) is |
| | given) as per Prostate Cancer Working Group 3 (PCWG3) criteria. |
| | All participants will undergo 2 PET/CT scans: one with the investigational |
| | agent [¹⁸F]CTT1057 and another with [⁶⁸Ga]Ga-PSMA-11 (as a component |
| | of the CTS Level 2 and for a secondary endpoint of assessment of |
| | concordance between the 2 PET/CT scans for detection of lesions at patient |
| | level). The 2 PET imaging procedures will be performed at least 14 days |
| | apart, and the PET/CT scan sequence for each participant will be assigned at |
| | random in a 1:1 ratio. |
| Study | Male participants ≥18 years of age, with biopsy proven prostate |
| population | adenocarcinoma and rising PSA after definitive therapy with prostatectomy |
| | or radiation therapy (external beam or brachytherapy), diagnosed of |
| | biochemically recurrent PCa. Approximately 190 participants will be |
| | enrolled to ensure at least 152 participants to complete the [¹⁸F]CTT1057 |
| | PET/CT scan procedure (i.e. investigational imaging agent administration |
| | and successful completion of the PET/CT scan), which will be required for |
| | the calculation of the co-primary endpoints. |
| Inclusion | Signed informed consent must be obtained prior to participation in the |
| criteria | study |
| | Biopsy proven prostate adenocarcinoma. |
| | Biochemical recurrence defined by American Urological Association |
| | (AUA) criteria for patients who have undergone radical prostatectomy |
| | (detectable or rising PSA value measured 6-13 wk after surgery, that is ≥0.2 |
| | ng/ml with a second determination of a PSA level >0.2 ng/mL at |
| | least 2 week apart) and by American Society for Radiation Oncology |
| | (ASTRO)-Phoenix criteria for patients who have undergone curative- |
| | intent radiotherapy (PSA ≥2 ng/mL above the nadir). |
| | Eastern Cooperative Oncology Group (ECOG) performance status 0-2 |
| | Participants must be adults ≥18 years of age |
| Exclusion | Inability to complete the needed investigational and standard-of-care |
| criteria | imaging examinations due to any reasons (severe claustrophobia, |
| | inability to lie still for the entire imaging time, etc.) |
| | Any additional medical condition, serious intercurrent illness, |
| | concomitant cancer or other extenuating circumstance that, in the opinion |
| | of the Investigator, would indicate a significant risk to safety or impair |
| | study participation, including, but not limited to, current severe urinary |
| | incontinence, hydronephrosis, severe voiding dysfunction, need of |
| | indwelling/condom catheters, New York Heart Association class III or IV |
| | congestive heart failure, history of congenital prolonged QT syndrome, |
| | uncontrolled infection, active hepatitis B or C, and Coronavirus Disease |
| | 2019 (COVID-19) |
| | Prior major surgery undergone less than 12 weeks prior to screening (with |
| | the exception of any surgery related to prostatic cancer) |
| | Known allergy, hypersensitivity, or intolerance to [¹⁸F]CTT1057, |
| | [⁶⁸Ga]Ga-PSMA-11, or to CT contrast |
| | Prior and current use of PSMA targeted therapies |
| | Prior and current treatment with Luteinizing Hormone-Releasing |
| | Hormone (LHRH) analogues |
| | Any prior ADT (first or second generation) within 9 months before |
| | screening |
| | Any 5-alpha reductase inhibitors within 30 days before screening |
| | Use of other investigational drugs within 30 days before screening |
| | Castration-resistant patients |
| | Patients with small cell or neuroendocrine PCa in more than 50% of |
| | biopsy tissue |
| Study | The term “Study Treatment” indicates the administration of at least one of |
| treatment | the two PET imaging agents: [¹⁸F]CTT1057 and [⁶⁸Ga]Ga-PSMA-11 |
| | independently of whether the PET/CT scans were acquired or not |
| Treatment of | In this study, the investigational imaging agent of interest is [¹⁸F]CTT1057, |
| interest | injected as a single intravenous dose of approximately 370 Mega- |
| | Becquerel (MBq) and subsequent PET/CT scan. |
| Efficacy | Hierarchical CTS with 3 levels: |
| assessments | Level 1: Histopathology assessments |
| | Level 2: Imaging diagnostic assessments: at least a high-resolution CT |
| | with contrast and a [⁶⁸Ga]Ga-PSMA-11 PET/CT. Other SoC imaging |
| | modalities if clinically indicated. Imaging data will be read centrally |
| | Level 3: PSA assessments |
| | [¹⁸F]CTT1057 PET/CT assessments, read centrally |
| Key safety | Adverse Events (AEs) |
| assessments | Serious Adverse Events (SAEs) |
| | Vital signs, physical examinations |
| | Electrocardiograms (ECGs) |
| | Laboratory parameters including hematology, clinical chemistry |
| | Concomitant medications and/or therapies |
| Other | Patient management questionnaire for secondary endpoint to evaluate the |
| assessments | change in patient management plans attributed to the PET/CT scan |
| Data analysis | The following data analyses are planned for the study: |
| | The co-primary endpoints of the study are region-level CLR and patient-level |
| | Positive Predictive Value (PPV) |
| | Region-level CLR is defined as the proportion of regions containing at |
| | least one true positive (TP) lesion (exactly localized correspondence |
| | between PET imaging and the reference standard), regardless of any co- |
| | existent false positive (FP) findings within the same region, out of all |
| | regions containing at least one PET-positive finding. |
| | Region-level CLR and its 95% confidence interval (CI) will be calculated |
| | using logistic random-effects models taking into account the correlation |
| | among regions within one patient. The lower bound of the 95% CI for CLR |
| | should be greater than 0.5 to attain the first co-primary endpoint. |
| | Patient-level PPV is defined as the proportion of patients who have at |
| | least one TP lesion (exactly localized correspondence between PET |
| | imaging and the reference standard), regardless of any co-existent FP |
| | findings, out of all patients who are PET/CT scan positive. Patient-level |
| | PPV and its 95% CI will be calculated based on the binomial distribution. |
| | The lower bound of the 95% CI for patient-level PPV should be greater |
| | than 0.2 to attain the second co-primary endpoint. |
| | Analyses for co-primary endpoints will be based on the efficacy analysis set |
| | (EFF). |
| | Centralized imaging assessments (CTS Level 2) will be used as reference |
| | standard if pathology (CTS Level 1) is not available, inconclusive or |
| | negative. |
| | Other secondary endpoints including patient-level sensitivity, patient-level |
| | specificity, patient-level negative predictive value, patient-level accuracy, |
| | patient-level correct detection rate, patient-level detection rate, |
| | [¹⁸F]CTT1057 scan inter-reader agreement, [¹⁸F]CTT1057 scan intra-reader |
| | agreement, change in patient management plans, region-level sensitivity, |
| | region-level specificity, region-level negative predictive value, region-level |
| | accuracy, concordance between [¹⁸F]CTT1057 and [⁶⁸Ga]Ga-PSMA-11 will |
| | also be analyzed. Detailed statistical methodology for these analyses will be |
| | provided in the statistical analysis plan (SAP). |

### Study Design

This is a prospective, open-label, multi center, single-arm Phase III study to evaluate the diagnostic performance of [¹⁸F]CTT1057 as a PET imaging agent for detection and localization of PSMA positive tumors in prostate patients diagnosed with biochemical recurrence (BCR) after radical prostatectomy or radiotherapy, using a composite truth standard as reference.

Approximately 190 participants will be enrolled to ensure at least 152 participants to complete the [¹⁸F]CTT1057 for PET/CT scan imaging, which will be read in a central Contract Research Organization (CRO) by 3 independent nuclear medicine physicians who will be blinded to any other patient data.

The CTS to be used as reference will be hierarchical in nature, with 3 levels of SoT procedures, that will be applied as follows:

### Level 1)

Histopathology if available (from prospective biopsy or salvage surgery performed within 8 weeks after the [¹⁸F]CTT1057 PET/CT scan); OR in case that histopathology is not available, inconclusive or negative:

### Level 2)

Imaging diagnostic procedures performed on each patient as clinically indicated per SoC, which must include at least a high resolution CT scan with contrast and a [⁶⁸Ga]Ga-PSMA-11 PET/CT performed within 8 weeks (either before or after) the [¹⁸F]CTT1057 PET/CT scan. Three-month follow-up imaging (from baseline) will also be used as part of the CTS level 2 in cases where it is clinically required for the diagnosis of particular lesion(s); OR if neither of the two above are feasible or deemed appropriate:

### Level 3)

50% or greater decline in PSA following radiation therapy (as long as no concomitant androgen deprivation therapy (ADT) is given) as per PCWG3 criteria (Scher et al 2016 Trial Design and Objectives for Castration-Resistant Prostate Cancer: Updated Recommendations From the Prostate Cancer Clinical Trials Working Group 3. J Clin Oncol; 34(12): 1402-18).

In the cases where pathology will be available, assessments by the local pathologists will be performed as per SoC, and results should be available within 2 weeks after surgery. Pathologists will be blinded to any PSMA-PET data (i.e. both PET/CT scans). In this case, only pathology will be used as SoT, so the imaging procedures will not be used for the co-primary endpoint calculations.

In the cases where pathology will not be available, the central read result of the imaging diagnostic procedures to be performed on each participant for the CTS level 2) will be used as Standard of Truth for the co-primary endpoint calculations.

All patients will undergo a [⁶⁸Ga]Ga-PSMA-11 PET/CT as part of the study, for both the CTS Level 2 (in case it is required as standard of truth) and the secondary endpoint of assessment of concordance between [¹⁸F]CTT1057 and [⁶⁸Ga]Ga-PSMA-11 for detection of lesions at patient level.

In addition to central review, local review of SoC images (including [⁶⁸Ga]Ga-PSMA-11) will be performed to be used by the treating physician/Clinical Study Investigator for patient management decisions and overall assessment.

A questionnaire on the planned patient management will be filled-in by the treating physician/Clinical Study Investigator before (questionnaire 1) and within 14 days after (questionnaire 2) knowing the results of the [¹⁸F]CTT1057 PET/CT scan. A local review of [¹⁸F]CTT1057 PET/CT images will also be performed by a local nuclear medicine physician or radiologist with expertise in reading oncology PET/CT scans and the results will be provided to the treating physician/Clinical Study Investigator for completion of questionnaire 2. Options will be given in the questionnaire to capture possible management plan, such as a) Surgery, b) Radiation alone, c) Radiation plus ADT, d) ADT alone, e) Observation/surveillance, f) Other (free text box). Any change in patient management plan between the questionnaire 1 and questionnaire 2 should not be based only on [¹⁸F]CTT1057 PET/CT scan results since this is an investigational diagnostic imaging product. Other diagnostic procedures should be performed as per SoC in order to confirm and implement the changed management plan.

### Screening Period

Written informed consent form (ICF) must be obtained prior to any screening procedures. The participant must be registered in the Interactive Response Technology (IRT) for screening. All procedures described in the Assessment Schedule must be carried out, prioritizing laboratory and imaging assessments to allow time to obtain the results at least 14 days prior the planned first PET imaging day (Day 1). Eligibility must then be confirmed at the latest on Day −14. The screening period should last up to 28 days.

Once eligibility is confirmed, the participants will be randomized in IRT to be assigned to one of the following two PET/CT scan sequences at random in a 1:1 ratio:
Sequence 1: [¹⁸F]CTT1057 on Day 1 (investigational imaging agent of interest) followed by [⁶⁸Ga]Ga-PSMA-11 at least 14 days apart (as part of CTS if required, and for secondary endpoint)
Sequence 2: [⁶⁸Ga]Ga-PSMA-11 (as part of CTS if required, and for secondary endpoint) on Day 1 followed by [¹⁸F]CTT1057 (investigational imaging agent of interest) at least 14 days apart

### PET Imaging Days

The 2 PET imaging procedures will be done at least 14 days apart. The day of the first PET imaging agent injection will be considered study Day 1.

### Study Design

A central read of the [¹⁸F]CTT1057 PET/CT scans will be performed by three independent nuclear medicine physicians or radiologists experienced in reading PET, who will be blinded to patient data, including the clinical condition of the patient, results of histopathology/biopsy, results of conventional imaging and PSA levels. Patients and regions will be graded on a two-point scale by each reader (0=negative; 1=positive). The 3 PET readers' results will be individually compared to the SoT to generate per-reader performance. An individual PET reader will be considered successful if he/she meets the predefined thresholds for both co-primary endpoints, and at least two of three readers should be successful for overall study positivity.

The criteria to be applied for PET positivity is the following:
A patient will be judged positive if at least one lesion in any region (i.e. prostate bed, Pelvic Lymph Node (PLN), skeleton, and other distant sites (extra-pelvic lymph nodes and viscera)) is visually positive.
A region will be judged as positive if at least one lesion in the region is visually positive.
Visually PET positive lymph nodes will be considered greater than blood pool (adjacent or mediastinal blood pool)
PET positive bone lesions will be considered greater than physiologic bone marrow

PET positive prostate, prostate bed and visceral lesions will be considered greater than physiologic background activity of the involvement organ or anatomic site, as previously described (Eiber et al 2015 Evaluation of Hybrid ⁶⁸Ga-PSMA Ligand PET/CT in 248 Patients with Biochemical Recurrence After Radical Prostatectomy. J Nucl Med; 56(5):668-74, Ceci et al 2015 (68)Ga-PSMA PET/CT for restaging recurrent prostate cancer: which factors are associated with PET/CT detection rate? Eur J Nucl Med Mol Imaging; 42:1284-94, Fendler et al 2019 Assessment of 68Ga-PSMA-11 PET Accuracy in Localizing Recurrent Prostate Cancer: A Prospective Single-Arm Clinical Trial. JAMA Oncol; 5(6):856-63

Consistency of the PET scan interpretation both between different readers and within readers is an important issue in medical imaging, as it affects portability of results between institutions and may affect patient care. The degree of inter- and intra-reader variability in the qualitative assessment of [¹⁸F]CTT1057 PET/CT images will be assessed as a secondary endpoint to ensure consistency of interpretation and hence reliable diagnosis, which has pivotal role in the patient management.

All patients will undergo 2 PET/CT scans: a [¹⁸F]CTT1057 PET/CT scan (investigational imaging agent) and a [⁶⁸Ga]Ga-PSMA-11 PET/CT scan (as part of the CTS level 2 if required, and for the secondary endpoint of assessment of concordance between [¹⁸F]CTT1057 and [⁶⁸Ga]Ga-PSMA-11 for detection of lesions at patient level). The two scans will be performed at least 2 weeks apart for each participant in order to ensure a clean safety profile assessment for each PET imaging radiopharmaceutical. Moreover, in order to counter-balance any potential change in lesions between the 2 PET/CT scans, the PET/CT scan sequence for each patient will be assigned at random, in a 1:1 ratio after enrolment.

### Rationale for Dose/Regimen and Duration of Treatment

For PET diagnostic radiopharmaceuticals, only a single administration is required, usually intravenously. The investigational PET radiopharmaceutical [¹⁸F]CTT1057 will be administered accordingly, as a single intravenous (i.v.) dose of approximately 370 MBq (266-407 MBq). This dose was shown to be safe and well tolerated in the Phase I study (Behr et al 2019 Phase I Study of CTT1057, an 18F-Labeled Imaging Agent with Phosphoramidate Core Targeting Prostate-Specific Membrane Antigen in Prostate Cancer. J Nucl Med; 60(7):910-6), and it is in line with the recommended dose of the commercial product [18F]FDG according to both the European Association of Nuclear Medicine (EANM) and Society of Nuclear Medicine guidelines (Delbeke D, Coleman RE, Guiberteau MJ, et al (2006) Procedure guideline for tumor imaging with 18F-FDG PET/CT 1.0. J Nucl Med; 47(5):885-95, Boellaard R, Delgado-Bolton R, Oyen WJG, et al (2015) FDG PET/CT: EANM procedure guidelines for tumour imaging: version 2.0. Eur J Nucl Med Mol Imaging; 42:328-54). Human dosimetry was studied in the Phase I study. The effective dose was estimated at 0.023±0.007 mSv/MBq, which is in line as well with the effective dose of the commercial product [18F]FDG (0.019 mSv/MBq) according to the EANM guideline (Boellaard R, Delgado-Bolton R, Oyen WJG, et al (2015) FDG PET/CT: EANM procedure guidelines for tumour imaging: version 2.0. Eur J Nucl Med Mol Imaging; 42:328-54) and in other publications (0.020-0.025 mSv/MBq) (Kaushik A, Jaimini A, Tripathi M, et al (2015) Estimation of radiation dose to patients from (18) FDG whole body PET/CT investigations using dynamic PET scan protocol. Indian J Med Res; 142:721-31), as well as the effective dose of other PSMA PET agents (Behr SC, Aggarwal R, VanBrocklin HF, et al (2019) Phase I Study of CTT1057, an 18F-Labeled Imaging Agent with Phosphoramidate Core Targeting Prostate-Specific Membrane Antigen in Prostate Cancer. J Nucl Med; 60(7):910-6). The radiation dose estimated from an i.v. administration of 370 MBq of [18F]CTT1057 is 8.51 mSV. Moreover, this dose allowed to obtain the optimal image quality, which was rated 76±5.4 on a Visual Analog Scale (VAS) of 1 to 100 (1=nondiagnostic, 100=perfect study), by 2 experienced Nuclear Medicine physicians (Behr SC, Aggarwal R, VanBrocklin HF, et al (2019) Phase I Study of CTT1057, an 18F-Labeled Imaging Agent with Phosphoramidate Core Targeting Prostate-Specific Membrane Antigen in Prostate Cancer. J Nucl Med; 60(7):910-6).

### Risks and Benefits

The use of PSMA-PET scanning of prostate cancer participants has been ongoing since 2011, mostly with [⁶⁸Ga]Ga-PSMA-11, to assess disease burden in the setting of both biochemical recurrence (BCR) and advanced/metastatic disease. Publications that report clinical use have demonstrated better sensitivity and specificity than choline-based PET imaging for PCa, with a very low rate of adverse events. [⁶⁸Ga]Ga-PSMA-11 has been shown to be well tolerated with no adverse events following infusion in a retrospective analysis of 1007 participants (Afshar-Oromieh A, Avtzi E, Giesel FL, et al (2015) The diagnostic value of PET/CT imaging with the (68)Ga-labelled PSMA ligand HBED-CC in the diagnosis of recurrent prostate cancer. Eur. J. Nucl. Med. Mol. Imaging; 42:197-209). Recently, [⁶⁸Ga]Ga-PSMA-11 has been approved in the USA (December 2020) as a radioactive diagnostic PET imaging agent in men with prostate cancer with suspected metastasis who are candidates for initial definitive therapy, or with suspected recurrence based on elevated serum PSA level ([⁶⁸Ga]Ga-PSMA-11 USPI). Subsequently, other PSMA-PET agents have been investigated and are under clinical development, all of them showing a good safety and tolerability profile. Prostate cancer patients diagnosed of localized disease, at primary staging, have been enrolled in studies requiring histopathology comparisons to determine diagnostic yield of the technique. A Phase-I study of [¹⁸F]CTT1057 in 20 prostate cancer patients (n=5 primary staging, n=15 mCRPC (NCT02916537) has shown an acceptable safety profile, without any radiotracer-related adverse reactions. The biodistribution of [¹⁸F]CTT1057 in humans is similar to that of other PSMA-targeted agents, and exposure rates of [¹⁸F]CTT1057 are also similar to those of the urea-based PET compounds, with the advantageous exception of lower exposure to kidneys and salivary glands. Preclinical work, dosimetry studies, and clinical experience with [¹⁸F]CTT1057 suggest good imaging quality properties and a favorable safety profile (Behr SC, Aggarwal R, VanBrocklin HF, et al (2019) Phase I Study of CTT1057, an ¹⁸F-Labeled Imaging Agent with Phosphoramidate Core Targeting Prostate-Specific Membrane Antigen in Prostate Cancer. J Nucl Med; 60(7):910-6).

As this is a study on the diagnostic performance of an investigational PET agent, patients enrolled are not expected to derive direct benefit. It is expected that distant disease will be identified in some patients as a consequence of this study and these patients may benefit from a more appropriated management plan, which will not be based on the investigational procedure alone, but confirmed by SoC diagnostic procedures. The risk-benefit ratio is expected to be favorable to the [¹⁸F]CTT1057 imaging agent.

Any risk to participants in this trial is minimized by compliance with the eligibility criteria and study procedures, as well as close clinical monitoring. Appropriate eligibility criteria are included in this protocol.

### Investigational and Control Drugs

### Description and Composition

The drug product [¹⁸F]CTT1057 370 MBq/mL solution for injection is a sterile ready-to use multidose solution containing [¹⁸F]CTT1057 as drug substance with a volumetric activity of 370 MBq/mL at reference date and time (calibration time). The natural decay of the radionuclide leads to a continuous decrease of the specific activity, the total radioactivity and the radioactive concentration of the drug product over time.

The radioactive drug substance is [¹⁸F]CTT1057, a fluorine (¹⁸F) labelled PSMA agent which is produced in an automated continuous process as an aqueous concentrated solution (so called Mother Solution). The mother solution is diluted further, taking into consideration the starting ¹⁸F activity, obtained radiochemical yield and targeted radioactivity concentration of 370 MBq/mL at the calibration time, to the finished product [18F]CTT1057 ready-to-use solution for injection. The composition of the finished product is shown in Table 2 below.

TABLE 2
Composition of [¹⁸F]CTT1057 370 MBq/mL solution for injection

| Components | Role |
|---|---|
| [¹⁸F]CTT1057 | Active substance |
| CTT1298 | Chemical precursor |
| NaCl | Isotonic agent |
| NaH₂PO₄ | Buffer |
| Na₂HPO₄ | Buffer |
| EtOH (excipient) | Eluent, Stabilizer against radiolysis |
| WFI | Solvent |

Ph. Eur = European Pharmacopeia, USP = United States Pharmacopeia
Note:
NaCl and WFI are components of NaCl 0.9% solution for injection and NaH₂PO₄ is used as a dihydrated salt form.

### Storage Condition

Store below 25° C. The shelf life is 10 hours after T₀ (T₀: time of activity measurement of the first quality control vial). 10 hours after T₀ corresponds to 6 hours after calibration time [T_{c}] (T_{c}=T₀₊₄ₕ).

### Investigational PET Imaging Agents

TABLE 3

| Investigational | | | |
|---|---|---|---|
| PET imaging | | | |
| agents | | | |
| (Name and | Pharmaceutical Dosage | Route of | |
| Strength) | Form | Administration | Supply Type |
| [¹⁸F]CTT1057 | Radiopharmaceutical | Intravenous use | Open label, vial or |
| | solution for injection | | syringe |
| 370 MBq/mL at | | | |
| Calibration time | | | |
| (Tc) | | | |
| [⁶⁸Ga]Ga- | Either provided as Kit for | Intravenous use | Open label, vial or |
| PSMA-11 | the radiopharmaceutical | | syringe |
| (150 MBq) | preparation of [⁶⁸Ga]Ga- | | |
| | PSMA-11 or as ready to | | |
| | use radiopharmaceutical | | |
| | solution for injection | | |

The [¹⁸F]CTT1057 radiopharmaceutical will be administered intravenously at a dose of approximately 370 MBq (range 266-407 MBq).
The [⁶⁸Ga]Ga-PSMA-11 radiopharmaceutical will be administered as a single intravenous (i.v.) dose of approximately 150 MBq. Administered doses must not be lower than 111 MBq or higher than 185 MBq in any case. The exact dose that will have been administered to each patient will be recorded in the CRF after measurement of the syringe both before and after administration in a dose calibrator.

The investigational imaging agent [¹⁸F]CTT1057 will be provided:
as a single mono-dose syringe (for US) or a single multidose vial (for European Union (EU)) ready to use radiopharmaceutical solution for injection, with a volumetric activity of 370 (±10%) MBq/mL at the reference date and time (calibration time (Tc)).

The natural decay of the radionuclide leads to a continuous decrease of the specific activity, the total radioactivity and the radioactive concentration (volumetric activity) over the time. Therefore the volume of the solution injected varies in order to provide the required amount of radioactivity at the date and time of injection.

The component [⁶⁸Ga]Ga-PSMA-11 will be provided:
as a kit for radiopharmaceutical preparation: single vial with a white lyophilized powder to be locally reconstituted with a solution of gallium-68 chloride (⁶⁸GaCl₃) in HCl eluted from an approved ⁶⁸Ge/⁶⁸Ga generator (for the clinical sites equipped with an approved ⁶⁸Ge/⁶⁸Ga generator).
as a single dose ready to use radiopharmaceutical solution: vial or syringe with the radiopharmaceutical solution supplied by the partner radiopharmacy (for the clinical sites not equipped with an approved ⁶⁸Ge/⁶⁸Ga generator).

The volume of [⁶⁸Ga]Ga-PSMA-11 solution for injection, corresponding to the radioactive dose to be administered, is calculated according to the estimated time of injection, on the basis of the current activity provided by the generator and of physical decay of the radionuclide (half-life=68 min). After reconstitution, the [⁶⁸Ga]Ga-PSMA-11 solution must be used according to instructions provided in the Pharmacy Manual.

1. A radioligand imaging agent, for use in a diagnostic method for determining the presence and/or localization of PSMA-positive tumors in a subject, particularly said subject is with prostate cancer and said PSMA-positive tumor is prostate cancer, wherein said subject has been diagnosed with biochemical recurrence, particularly said biochemical recurrence follows a radical prostatectomy or a radio-therapy, and wherein said radioligand imaging agent is a PSMA-binding compound comprising a phosphoramidate group and a [¹⁸F]-fluoro group.

2. The radioligand imaging agent for use according to claim 1, where said agent is a PSMA-binding compound of formula (I): 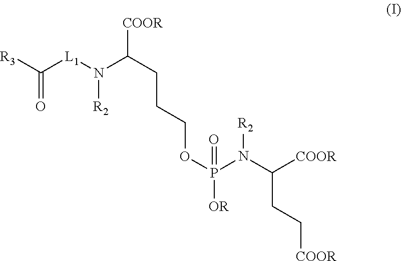
or any pharmaceutically acceptable salts thereof, wherein
each R is independently hydrogen or a protecting group, preferably t-butyl or benzyl,
each R2 is independently hydrogen or C1-C6 alkyl,
R3 is a phenyl or pyridyl, each substituted with [¹⁸F]-fluoro group and optionally substituted with a second group selected from the group consisting of halo, cyano and nitro,
and L1 is a linker, preferably comprising one or more groups selected from one or more amino-acids, C1-C18 alkylene, and heteroalkylene comprising 1 to 35 carbon atoms and 1 to 15 heteroatoms, said heteroalkylene being optionally substituted by one or more substituents selected from oxo and C1-C6 alkyl, and more preferably L1 is a linker selected from 1 to 6 amino-acids.

3. The radioligand imaging agent for use according to claim 1 or 2, where said radioligand imaging agent is a PSMA-binding compound of formula (II): 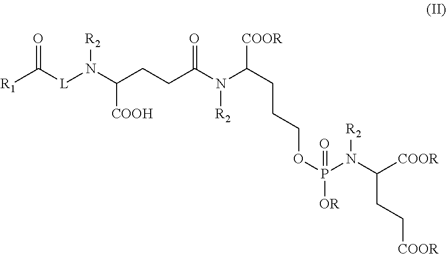
and any pharmaceutically acceptable salts thereof, wherein
L is a linker comprising a moiety of the formula —NH—CH₂CH₂—(OCH₂CH₂—)y—C(O)— or a group of the formula 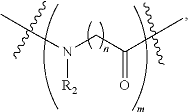
wherein y is 1, 2, 3, 4, 5, 6, 7, 8, 9, 10, 11, or 12:
m is 1, 2, 3, or 4;
each n is independently 1, 2, 3, 4, 5, 6, 7, 8, 9, 10, 11, or 12;
R1 is a phenyl or pyridyl, each substituted with [¹⁸F]-fluoro group and optionally substituted with a second group selected from the group consisting of halo, cyano and nitro;
each R2 is independently hydrogen or C1-C6 alkyl; and
each R is independently hydrogen or a protecting group (e.g t-butyl or benzyl).
Provided that when L is a group of the formula 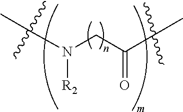
the combination of m and n result in a linear linker length of 3 to 21 atoms.

4. The radioligand imaging agent for use according to any one of claims 1-3, wherein said radioligand imaging agent is the PSMA-binding compound of formula (III): 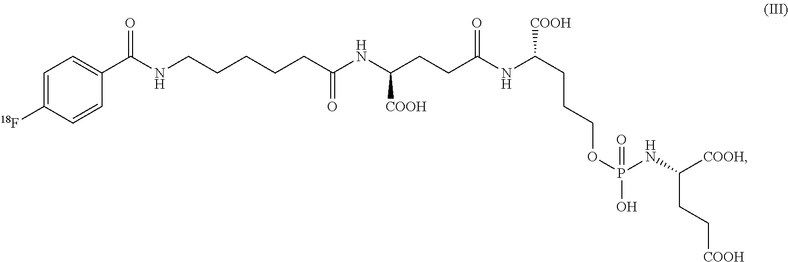 [SMILES: O=C(O)CC[C@H](NP(=O)(O)OCCC[C@H](NC(=O)CC[C@H](NC(=O)CCCCCNC(=O)c1ccc([18F])cc1)C(=O)O)C(=O)O)C(=O)O]
or any pharmaceutically acceptable salts thereof.

5. The radioligand imaging agent for use according to any one of claims 1-4, wherein said subject has been diagnosed with biochemical recurrence after radical prostatectomy or radiotherapy.

6. The radioligand imaging agent for use according to any one of claims 1-5, wherein said radioligand imaging agent is formulated as a solution for injection or infusion in a concentration providing a volumetric radioactivity from 150 to 1000 MBq/mL, for example 370 MBq/mL+/−10% at calibration time.

7. The radioligand imaging agent for use according to any one of claims 1-6, wherein said radioligand imaging agent is administered intravenously at an effective dose comprised between 250 and 450 MBq, typically about 370 MBq.

8. The radioligand imaging agent for use according to claim 6 or 7, wherein a first PET scan imaging of the subject is performed between 60 and 120 minutes post-injection, and optionally a second scan imaging is performed up to 180 minutes post-injection.

9. The radioligand imaging agent for use according to claim 1-8, wherein the method comprises:
[i] administering to said subject, an effective dose of a radioligand imaging agent as defined in any of claims 1-6,
[ii] imaging said subject by PET scan, either PET/CT or PET/MRI scan,
[iii] analysing the image obtained by the PET scan, and,
[iv] determining the presence and/or the localization of PSMA positive tumors in said subject.

10. A solution for injection or infusion, which is an aqueous solution comprising said radioligand imaging agent as defined in any of claims 1-5, in a concentration providing a volumetric radioactivity of from 150 to 1000 MBq/mL, for example about 370 MBq/mL, and one or more pharmaceutically acceptable excipients.

11. The solution of claim 10, in which the precursor compound of formula (IV) 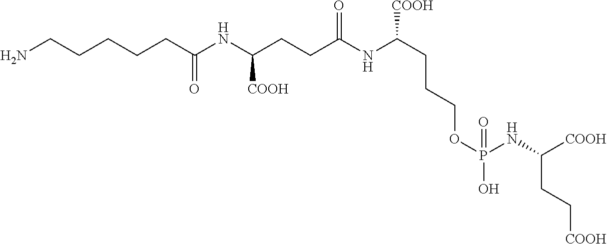 [SMILES: NCCCCCC(=O)N[C@@H](CCC(=O)N[C@@H](CCCOP(=O)(O)N[C@@H](CCC(=O)O)C(=O)O)C(=O)O)C(=O)O]
is present in a concentration of not more than 5.0 μg/mL, preferably not more than 4.0 μg/mL, more preferably not more than 3.0 μg/mL, even more preferably not more than 2.0 μg/mL, even more preferably not more than 1.0 μg/mL.

12. The solution of claim 10 or 11, further comprising a buffer for a pH between 5.0 and 8.0, preferably between 6.0 and 8.0, more preferably between 6.5 and 7.5, preferably said buffer is a phosphate buffer, and an isotonic agent, preferably sodium chloride.

13. The solution of any one of claims 10-12, further comprising a stabilizer against radiolysis, preferably a stabilizer that is suitable as an eluent during the manufacturing of the solution, preferably said stabilizer and/or eluent is an alcohol, preferably ethanol.

14. The solution of claim 13, comprising
[i] NaCl in a concentration from 8.0 to 9.5 mg/mL; preferably from 8.6 to 8.9 mg/mL,
[ii] NaH₂PO₄ in a concentration from 0.03 to 0.3 mg/mL; preferably from 0.1 to 0.2 mg/mL;
[iii] Na₂HPO₄ in a concentration from 0.2 to 1.2 mg/mL; preferably from 0.3 and 1.1 mg/mL;
[iv] ethanol in a concentration from 5-50 mg/mL; preferably from 10.0 and 39.5 mg/mL; and,
[v] the precursor compound at a concentration of not more than 5.0 μg/mL preferably not more than 4.0 μg/mL, more preferably not more than 3.0 μg/mL, even more preferably not more than 2.0 μg/mL, even more preferably not more than 1.0 μg/mL.
